(19) United States
(12) Patent Application Publication
(10) Pub. No.: US 2025/0368721 A1
(43) Pub. Date: Dec. 4, 2025

(54) Nasally Administered Formulations for Reducing Allergic Symptoms and Methods of Using the Same

(71) Applicant: John E. Boyd, OLD GREENWICH, CT (US)

(72) Inventor: John E. Boyd, OLD GREENWICH, CT (US)

(21) Appl. No.: 19/299,666

(22) Filed: Aug. 14, 2025

Related U.S. Application Data
(63) Continuation of application No. 17/302,136, filed on Apr. 25, 2021.
(60) Provisional application No. 63/015,677, filed on Apr. 26, 2020.
(60) Provisional application No. 62/704,332, filed on May 5, 2020.
(60) Provisional application No. 62/706,204, filed on Aug. 5, 2020.

(51) Int. Cl. C07K 16/08; C07K 16/12

(52) U.S. Cl. CPC C07K 16/08; C07K 16/12

(57) ABSTRACT

Liquid formulation comprising a plurality of antibody structures that specifically bind to at least one allergen and adapted for administering to a subject nasally to reduce at least one symptom caused the least one allergen on the subject and methods of using the same.

### RELATED APPLICATIONS

This application is a continuation application of U.S. Non-Provisional application Ser. No. 17/302,136, filed Apr. 25, 2021, which claims the benefit of and priority to U.S. Provisional Application No. 63/015,677, entitled “Compounds, Compositions and Formulations for Disrupting or Destroying Pathogens and Methods of Using the Same”, filed Apr. 26, 2020; U.S. Provisional Application No. 62/704,332, entitled “Compounds, Compositions and Formulations for Disrupting or Destroying Pathogens and Methods of Using the Same”, filed May 5, 2020; and U.S. Provisional Application No. 62/706,204, entitled “Compounds, Compositions and Formulations for Disrupting or Destroying Pathogens and Methods of Using the Same”, filed Aug. 5, 2020, the entire contents of the U.S. non-provisional application and each U.S. provisional application are hereby incorporated by reference.

### TECHNICAL FIELD

The invention generally relates to compositions for destroying, disrupting, or inactivating pathogens (e.g., viruses) and/or allergens using antibodies and/or surface-active agents immobilized on microparticles or immobilized in gels, foams or pastes.

### BACKGROUND

Several publications are referenced in this application. The cited references describe the state of the art to which this invention pertains and are each hereby incorporated by reference in its entirety, particularly the compositions and methods set forth in the detailed description and figures of each reference.

Viruses are the smallest of parasites (as currently known) and are completely dependent on the cells they infect for their reproduction. Viruses are typically composed of an outer coat of protein, which is sometimes surrounded by a lipid envelope or membrane, and an inner nucleic acid core including either RNA or DNA. Typically, after docking with the cellular membrane of a susceptible cell, the viral core penetrates the cell membrane to initiate the viral infection. After infecting the cell, the virus commandeers the cell's molecular machinery to direct the virus's own replication. The “replicative phase” of the viral lifecycle may begin immediately upon entry into the cell, or may occur after a period of dormancy or latency. After the infected cell synthesizes sufficient amounts of viral components, the “packaging phase” of the viral life cycle begins and new viral particles are assembled. Some viruses reproduce without killing their host cells, and many of these bud from host cell membranes. Other viruses cause their host cells to lyse or burst, releasing the newly assembled viral particles into the surrounding environment, where they can begin the next round of their infectious cycle. Several hundred different types of viruses are known to infect humans, animals and plants. Viruses that primarily infect humans are spread mainly via respiratory and enteric excretions.

Of these viruses that infect humans, many infect their hosts without producing overt symptoms, while others (e.g., influenza) produce a well-characterized set of symptoms. Importantly, although symptoms can vary with the virulence of the infecting strain, identical viral strains can have drastically different effects depending upon the health and immune response of the host. Despite remarkable achievements in the development of vaccines for certain viral infections (i.e., polio and measles), and the eradication of specific viruses from the human population (e.g., smallpox), viral diseases remain as important medical and public health problems. Indeed, viruses are responsible for several “emerging” (or reemerging) diseases (e.g., West Nile encephalitis & Dengue fever), and also for the largest pandemic in the history of mankind (HIV and AIDS).

An outbreak of a virulent respiratory virus, now known as Severe Acute Respiratory Syndrome (SARS), was identified in Hong Kong, China and a number of other countries around the world in 2003. Patients typically had symptoms including fever, dry cough, dyspnea, headache, and hypoxemia. Isolates of the SARS virus appear to have homology with at least the RNA polymerase gene of several known coronaviruses. In 2019, another outbreak of a virulent respiratory virus, now known as severe acute respiratory syndrome coronavirus-2 (SARS-CoV-2 and often abbreviated as COVID-19), was identified and spread around the world in 2019/2020. Patients typically had symptoms including fever, dry cough, and shortness of breath.

As of Apr. 21, 2020, more than 2.53 million cases have been reported across 185 countries and territories, resulting in more than 174,000 deaths. As of Jul. 27, 2020, the numbers rose to more than 16 million cases have been reported across 188 countries and territories, resulting in more than 650,000 deaths. As of Apr. 12, 2021, the numbers rose to more than 136 million cases have been reported across 192 countries and territories, resulting in more than 2,944,366 deaths.

It is believed the SARS-CoV-2 virus—the coronavirus that causes COVID-19—infects the nasal cavity to a great degree—replicating specific cell types—and infects and replicates progressively less well in cells lower down the respiratory tract, including the lungs; it is believed the virus tends to become firmly established first in the nasal cavity, but in some cases the virus is aspirated into the lungs, where it may cause more serious disease, including potentially fatal pneumonia. See, SARS-CoV-2 Reverse Genetics Reveals a Variable Infection Gradient in the Respiratory Tract, Hou et al., Cell 182, pp. 429-446, Jul. 23, 2020. It is also believed that infected individuals with high viral loads can become “superspreaders” (see, “Do superspreaders generate new superspreaders? A hypothesis to explain the propagation pattern of COVID-19”, by Pablo M. Beldomenico, International Journal of Infectious Diseases 96 (2020) 461-463).

Bacteria are unicellular microorganisms (made up of one cell) and also have a membrane, specifically a very thin cell membrane (approximately 8 nm thick) that covers the whole body of the bacteria. The cell membrane separates the inner fluids of the cell, called cytoplasm, from the surrounding environment. The cell membrane is also referred to as the cytoplasmic membrane. The cell membrane is a selective barrier, allowing useful molecules to enter and waste material to exit from the cell. Phospholipid molecules are the building block of the bacteria cell membrane. The membrane comprises millions of these phospholipid molecules lying side by side forming a phospholipid bilayer. If the bacteria cell membrane breaks, the contents inside the cell leak out and the bacteria dies.

There is a growing need for prophylactic or therapeutic treatments against the COVID-19 and other past and future viruses, bacterium and other pathogenic microorganisms and also for allergens and other substances.

### SUMMARY OF INVENTION

The invention relates to compounds, complexes, compositions and formulations for disrupting, destroying, and/or inactivating pathogens (e.g., virus, bacteria, etc.) and/or allergens, and to methods of using the same.

One aspect of the invention relates to destroying, disrupting or inactivating a pathogen using antibodies and/or surface-active agents (e.g., amphiphile molecules) and methods of using the same, preferably for destroying or disrupting or inactivating a pathogen membrane (e.g., virus membrane or bacterial membrane).

Another aspect of the invention relates to a therapeutic composition for treatment of an illness and/or symptoms caused by a pathogen (e.g., virus, bacteria) and/or a foreign substance (e.g., allergen), the composition comprising at least one particle and at least one antibody structure comprising an antibody or antibody fragment that specifically binds to the pathogen and/or foreign substance (e.g., allergen), wherein the at least one antibody structure is attached to, connected to, adsorbed onto, absorbed into, or embedded in the particle, wherein the particle has an aerodynamic diameter of greater than 10 μm and less than 100 mm, preferably less than 200 μm, and/or the therapeutic composition is an environmentally compatible and/or biocompatible composition essentially nontoxic to human, animal and/or plant life.

Another aspect of the invention relates to compositions for destroying, disrupting or inactivating a pathogen membrane (e.g., virus membrane) using antibodies and/or surface-active agents (e.g., amphiphile molecules) and methods of using the same.

Another aspect of the invention relates to a therapeutic composition for treatment of an illness and/or symptoms caused by a pathogen (e.g., virus, bacteria), the therapeutic composition comprising at least one amphiphile molecule and at least one antibody structure comprising an antibody or antibody fragment that specifically binds to the pathogen (e.g., virus, bacteria).

Another aspect relates to therapeutic compositions for destroying, disrupting or inactivating the pathogen (e.g., virus) and/or the foreign substance (e.g., allergen) using antibodies attached to microparticles and methods of using the same.

Another aspect relates to a liquid formulation comprising a therapeutic composition as described herein, wherein the therapeutic composition is dispersed in a liquid, wherein liquid formulation is configured for administering to a subject topically, by injection, to the eye, orally, by inhalation and/or otherwise (e.g., by injection, dermally (e.g., patch), sublingually, suppository, etc.)

Another aspect relates to an aerosol comprising the therapeutic composition as described herein, wherein the therapeutic composition is dispersed in an aerosol or dispersible liquid, wherein the aerosol or liquid formulation is configured for administering to a subject via nasal passages (i.e., intranasal administration) and/or orally.

Another aspect relates to methods of inhibiting viral illness and/or symptoms comprising administering to a subject in need thereof a therapeutically effective amount of a therapeutic composition of the invention as described herein.

Another aspect relates to methods of inhibiting illness (e.g., viral illness) and/or symptoms comprising administering to a subject in need thereof a therapeutically effective amount of the therapeutic compositions of the invention as described herein.

The foregoing has outlined some of the aspects of the present invention. These aspects should be construed strictly as illustrative of some of the more prominent features and applications of the invention, rather than as limitations on the invention. Many other beneficial results can be obtained by modifying the embodiments within the scope of the invention. Accordingly, for other objects and a full understanding of the invention, refer to the summary of the invention, the detailed description describing the preferred embodiment in addition to the scope of the invention defined by the claims and the accompanying drawings. The unique features characteristic of this invention and operation will be understood more easily with the description and drawings. It is to be understood that the drawings are for illustration and description only and do not define the limits of the invention.

### BRIEF DESCRIPTION OF THE DRAWINGS

The above-mentioned and other features of the inventions disclosed herein are described below with reference to the drawings of the preferred embodiments. The illustrated embodiments are intended to illustrate, but not to limit the inventions. The drawings contain the following figures:

FIG. 1 is a graphical representation of an active complex according to one embodiment of the invention and a COVID-19 virus bound to the active complex.

FIG. 2 is a graphical representation of an active complex including a surface-active agent according to another embodiment of the invention and viruses (including two inactivated viruses and a virus bound to the active complex).

FIG. 3(a) is a graphical representation of an active complex according to another embodiment of the invention (without a particle). FIG. 3(b) is a graphical representation of an active complex according to another embodiment of the invention including a particle.

FIG. 4 is a graphical representation of an active complex according to another embodiment of the invention showing a concave particle bound to a cellular surface.

FIG. 5 is a graphical representation of an active complex according to another embodiment of the invention showing a support comprising an assemblage of nanofibers bound to a cellular surface.

FIG. 6(a) is a graphical representation of an active complex including a flexible support according to another embodiment of the invention. FIG. 6(b) is a graphical representation of FIG. 6(a) showing the support as bent and resulting destruction of the bound virus.

FIG. 7(a) is a graphical representation of an active complex including a flexible support according to another embodiment of the invention. FIG. 7(b) is a graphical representation of FIG. 7(a) showing the bending/twisting of the support and resulting destruction of the bound virus.

FIG. 8 is a graphical representation of an endotracheal tube according to one embodiment of the invention.

FIG. 9 is a graphical representation of a wound dressing comprising a therapeutic composition according to one embodiment of the invention.

### DETAILED DESCRIPTION OF PREFERRED EMBODIMENTS

In the following description, for purposes of explanation, specific details are set forth in order to provide a thorough understanding of different aspects of the present invention. It will be evident, however, to one skilled in the art that the present invention as defined by the claims may include some or all of the features or embodiments herein described and may further include obvious modifications and equivalents of the features and concepts described herein.

### Definitions

As used herein, the singular forms “a”, “an” and “the” include plural referents unless the context clearly dictates otherwise. Thus, for example, reference to “a molecule” includes aspects having two or more such molecules unless the context clearly indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect and “about” is utilized herein to represent an inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue. It will be further understood that the endpoints of each of the ranges are significant both in relation to the other endpoint, and independently of the other endpoint.

Terms used herein, such as “aspect” or “embodiment” or “exemplary” or “exemplified,” are not meant to show preference, but rather to explain that the aspect discussed thereafter is merely one example of the aspect presented.

Additionally, as used herein, relative terms, such as “substantially”, “generally”, “approximately”, and the like, are utilized herein to represent an inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

The term “allergen” as used herein means a substance that causes an allergic reaction in a human or animal, including pollen, mold, dust, dust mite parts and/or excretions, and pet dander.

The term “antibody” or “antibodies” is used in the broadest sense and specifically covers, for example, single monoclonal antibodies (including agonist, antagonist, and neutralizing antibodies), antibody compositions with polyepitopic specificity, polyclonal antibodies, single chain antibodies, multi-specific antibodies (e.g., bispecific antibodies), immune-adhesins, and fragments of antibodies (defined below) as long as they exhibit the desired biological or immunological activity. The term “immunoglobulin” (Ig) is used interchangeable with antibody herein.

The term “antibody fragments” is used in the broadest sense and specifically covers, for example, a portion of a full-length antibody, generally the antigen binding or variable region thereof. Examples of antibody fragments include Fab, Fab′, F(ab′)2, and Fv fragments; single-chain antibody molecules; diabodies; linear antibodies; and multi-specific antibodies formed from antibody fragments.

“Bifunctional linking reagent” or “bifunctional linkers” refers to a molecule with one functional group reacting with a chemical moiety on a first molecule and a second functional group reacting with a chemical moiety on a second molecule. Bifunctional linking reagents can be used to link two different molecules via such functional groups.

The term “cellular membrane” as used herein refers to a biological membrane enclosing or separating structure acting as a selective barrier, within or around a cell (e.g., human cell or bacteria or virus) or an emergent viral particle. The cellular membrane is typically selectively permeable to ions and organic molecules and controls the movement of substances in and out of cells. The cellular membrane typically comprises a phospholipid uni- or bilayer, and optionally associated proteins and carbohydrates. As used herein, the cellular membrane refers to a membrane obtained from a naturally occurring biological membrane of a cell or cellular organelles, or one derived therefrom. As used herein, the term “naturally occurring” refers to one existing in nature. As used herein, the term “derived therefrom” refers to any subsequent modification of the natural membrane, such as isolating the cellular membrane, creating portions or fragments of the membrane, removing and/or adding certain components, such as lipid, protein or carbohydrates, from or into the membrane taken from a cell or a cellular organelle. A membrane can be derived from a naturally occurring membrane by any suitable methods. For example, a membrane can be prepared or isolated from a cell or a virus and the prepared or isolated membrane can be combined with other substances or materials to form a derived membrane. In another example, a cell or virus can be recombinantly engineered to produce “non-natural”substances that are incorporated into its membrane in vivo, and the cellular or viral membrane can be prepared or isolated from the cell or the virus to form a derived membrane.

The term “connected to” includes connected or linked directly or indirectly. Thus, for example, reference to a “molecule A connected to molecule B” includes aspects having molecule A and molecule B directly connected by a bond, or otherwise and also molecule A and molecule B indirectly connected by an intermediate structure (e.g., ligand or another linking molecule or moiety) or structure (e.g., “molecule A and molecule B are each independently connected to structure”) unless the context clearly indicates otherwise.

“Chimeric” refers to the combination of two molecules from different sources. A “chimeric molecule” is a bifunctional molecule. An example of a chimeric molecule is a viral-specific ligand that is modified to include a non-native domain, i.e., a bacterial-specific ligand. The molecules may be physically associated through a variety of means, including but not limited to, ionic bonds, covalent bonds or hydrophobic interactions.

A “domain” is a region of a molecule that has a defined functional attribute. Domains can refer to proteins, carbohydrates or lipids. The domains can be made in a variety of ways. Also, the domains can be derived from or homologous to naturally occurring molecules. Alternatively, the domains can be isolated from a library of molecules made up of polymers with sequences not occurring in nature. Examples of “domains” include a “viral-specific ligand” and a “bacterial-specific ligand”.

A “ligand” is a molecule which has the ability to bind to another molecule. A ligand can be any ion or molecule with binding properties. Examples of classes of ligands include, without limitation, ions, organic molecules, inorganic molecules, peptides, proteins, polypeptides, carbohydrates, lipids, and polymers.

The term “bacterial-specific ligand” refers to a molecule that interacts with and binds to, without limitation, a protein, carbohydrate or lipid on the surface, including the membrane or cell wall, of a bacterium. The binding is considered specific when more of the ligands binds to the target bacteria than to the background of mucosa, for example. Bacterial-specific ligands may also be isolated from combinatorial peptide libraries or from libraries comprised of nucleic acid sequences from bacteria, mammals, viruses, or plants. Bacterial-specific ligands can be antibodies (e.g., single chain antibodies, Fab, and other antibody fragments), peptides, and small organic molecules. Essentially, bacterial-specific ligands can be identified or isolated from any source as long as the bacterial-specific ligand possesses the ability to bind to a bacterial molecule or bacterium. Bacterial-specific ligands can be organic and inorganic molecules. Such molecules may be identified through screening of a library.

The term “effective amount” refers to the amount of a therapy (e.g., a prophylactic or therapeutic agent, composition, formulation, treatment time, etc.), which is sufficient to reduce the severity, and/or duration of an infection or illness or disease, ameliorate one or more symptoms thereof, reduce the viral/bacterial load, prevent the advancement of an infection or illness or disease or symptoms, and/or cause regression of an infection/illness/disease/symptoms, and/or which is sufficient to result in the prevention of the development, recurrence, onset, or progression of an infection/illness/disease or one or more symptoms thereof, and/or enhance or improve the prophylactic and/or therapeutic effect(s) of another therapy (e.g., another therapeutic agent).

As used herein, the terms “optional” or “optionally” mean that the subsequently described event or circumstance may or may not occur or the component might be omitted, and that the description includes instances where the event or circumstance occurs and instances where it does not or when the component is present or not present.

The term “pathogen” means a virus, bacteria or other microorganism (e.g., archaea, fungi, mold, yeast, protozoa, algae, phage, etc.).

The term “pharmaceutically acceptable” means being approved by a regulatory agency of the Federal or a state government or listed in the U.S. Pharmacopia, European Pharmacopia or other generally recognized pharmacopia for use in animals, and more particularly in humans.

The term “pharmaceutical drug” as used herein refers to a medication or medicine used to cause a change in an organism's physiology or psychology when consumed or administered, or otherwise intended to produce a biological effect.

The terms “prevent”, “preventing”, and “prevention” refer to the prevention or reduction of the recurrence, onset, development or progression of an infection, illness and/or disease, or the prevention or reduction of the severity and/or duration of an infection, illness and/or disease or one or more symptoms thereof.

The terms “prophylactic agent” and “prophylactic agents” refer to any agent(s) which can be used in the prevention of an infection, illness and/or disease and/or one or more symptoms thereof, or reduce the impact of the infection, illness and/or disease on the patient.

The term “prophylactically effective amount” refers to the amount of a composition (e.g., liquid or aerosol formulation of the invention) which is sufficient to result in the prevention of the development, recurrence, onset or progression of an infection, illness and/or disease, and/or one or more symptoms thereof, or reduce the impact of the infection, illness and/or disease on the patient.

As used herein, a “protocol” includes dosing schedules and dosing regimens. The protocols herein are methods of use and include prophylactic and therapeutic protocols.

The term “respirable” as used herein refers to dry particles or dry powders that may enter into the lower respiratory tract (e.g., pulmonary delivery) in a subject by inhalation. Typical respirable dry powders or dry particles have a mass median aerodynamic diameter (MMAD) of less than about 10 microns, more typically about 5 microns or less. The term “non-respirable” as used herein refers to particles or powders or fibers or other structures that do not typically enter into the lower respiratory tract in a subject by inhalation because of size, shape, surface characteristics, composition, and/or other factors.

The terms “therapies” and “therapy” can refer to any protocol(s), method(s) and/or agent(s) that can be used in the prevention, treatment, management or amelioration of an infection, illness and/or disease, and/or one or more symptoms thereof. In certain embodiments, the terms “therapy” and “therapies” refer to biological therapy, and/or other therapies useful for the treatment of an infection, illness and/or disease known to medical personnel skilled.

The terms “treat”, “treating” and “treatment” refer to the reduction or amelioration of the progression, severity, and/or duration of an infection, illness and/or disease and/or reduces or ameliorates one or more symptoms of an infection, illness and/or disease. In specific embodiments, such terms refer to the reduction or inhibition of the replication of a virus, the inhibition or reduction in the spread of a virus (e.g., to other tissues or subjects), the inhibition or reduction of infection of a cell with a virus, or the amelioration of one or more symptoms associated with a virus infection.

As used herein, the term “surfactant” refers to organic substances having amphipathic structures; namely, they are composed of groups of opposing solubility tendencies, typically an oil-soluble hydrocarbon chain and a water-soluble ionic group. Surfactants can be classified, depending on the charge of the surface-active moiety, into anionic, cationic, and nonionic surfactants. Surfactants are often used as wetting, emulsifying, solubilizing, and dispersing agents for various pharmaceutical compositions and preparations of biological materials.

As used herein, “surface active agent” is a compound, molecule, molecules and/or complex of molecules having the ability to disrupt, destroy and/or otherwise inactivate pathogen envelopes or membranes and/or disrupt and/or destroy and/or inactivate the pathogen (e.g., viruses). In some examples, a surface-active agent may be a composition comprising a surfactant and one or more other agents such antibodies, chelating agents and preservatives.

The invention relates to compositions for destroying, disrupting and/or inactivating a pathogen (e.g., virus, preferably the virus membrane) or other foreign substance (e.g., allergen or irritant) using antibodies and/or surface-active agents including surfactants, and methods of using the same.

The practice of the present invention will employ, unless otherwise indicated, conventional techniques of nanotechnology, nano-engineering, molecular biology (including recombinant techniques), microbiology, cell biology, biochemistry, immunology, and pharmacology, which are within the skill of the art. Such techniques are explained fully in the literature, such as, Molecular Cloning: A Laboratory Manual, 2nd ed. (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds., 1987, and periodic updates); PCR: The Polymerase Chain Reaction (Mullis et al., eds., 1994); and Remington, The Science and Practice of Pharmacy, 20th ed., (Lippincott, Williams & Wilkins 2003), each hereby incorporated by reference in its entirety.

Many viruses include a lipid membrane outer shell. For example, the Covid-19 virus includes a lipid membrane shell that encloses the RNA/DNA enclosed in protein. According to the preferred embodiments of the invention, the compounds, compositions and methods are configured for the treatment of an enveloped pathogen, particularly an enveloped virus. Non-limiting examples of an industrially relevant virus include a retrovirus, a herpesvirus, a flavivirus, a poxvirus, a hepadnavirus, a hepatitis virus, an orthomyxovirus, a paramyxovirus, a rhabdovirus, or a togavirus. According to preferred embodiments of the invention, the virus is selected from the group consisting of an adenovirus, African swine fever-line virus, arenavirus, arterivirus, astrovirus, baculovirus, badnavirus, barnavirus, birnavirus, bromovirus, bunyavirus, calicivirus, capillovirus, carlavirus, caulimovirus, circovirus, closterovirus, comovirus, coronavirus (including SARS and COVID-19), cotricovirus, cystovirus, deltavirus, dianthovirus, enamovirus, filovirus, flavivirus, furovirus, fusellovirus, geminivirus, hepadnavirus, herpesvirus, hordeivirus, hypovirus, ideaovirus, inovirus, iridovirus, levivirus, lipothrixvirus, luteovirus, machlomovirus, marafivovirus, microvirus, myovirus, necrovirus, nodavirus, orthomyxovirus, papovavirus, paramyxovirus, partitivirus, parvovirus, phycodnavirus, picornavirus, plamavirus, podovirus, polydnavirus, potexvirus, potyvirus, poxvirus, reovirus, retrovirus, rhabdovirus, rhizidiovirus, sequevirus, siphovirus, sobemovirus, tectivirus, tenuivirus, tetravirus, tobamavirus, tobravirus, togavirus, tombusvirus, totivirus, trichovirus, tymovirus, and umbravirus.

An “enveloped virus” (or “enveloped pathogen) means a virus (or other pathogen) containing an envelope (e.g., a proteinaceous envelope) which surrounds the viral capsid (or pathogen center). For example, some simple viruses consist of only capsid protein surrounding nucleic acid, more complex viruses have these basic structures enveloped within a phospholipid membrane. This membrane, which is typically derived from a host cell, is pinched-off during viral release though a mechanism termed budding. Because these viral membranes are derived from the host, they may contain a complement of membrane-bound host cell proteins; other enveloped viruses exclude host cell proteins through a mechanism used by these viruses.

The inventions described herein are also applicable to treating illnesses, diseases and/or symptoms caused by other pathogens (e.g., bacteria, archaea, fungi, mold, yeast, protozoa, algae, phage, etc.) and/or foreign substances (e.g., irritant(s) or allergen(s) (e.g., pollen, dust mite parts and/or excretions, pet dander), etc.), specifically those pathogens having outer membranes such a bacteria or enveloped virus. However, particularly preferred embodiments relate to treatments for virus, particularly viruses such as respiratory viruses which start with infecting the upper respiratory system (e.g., nasal passages, throat).

Particularly preferred embodiments of the invention relate to viruses that are pathogenic to humans and/or animals, even more preferably pathogenic to humans. Alternative embodiments of the invention may be and/or also be applicable to viruses that are pathogenic to plants (e.g., using compositions (e.g., liquid, paste, spray) applied to portion(s) of plant where virus typically enters).

The invention relates to destroying and/or disrupting and/or inactivating (including expelling from host by swallowing and/or by cough or sneeze and/or flushing with liquid) one or more pathogens (e.g., virus, bacteria, etc.), or other foreign substance, using antibodies and/or surface-active agents (e.g., amphiphile molecules) and methods of using the same, preferably destroying or disrupting a pathogen membrane (e.g., virus membrane), including compounds, complexes, compositions and formulations for use in such destroying and/or disrupting and/or inactivating the pathogen, and also to methods of using the same.

According to preferred embodiments, the invention reduces the “load” of the pathogen (e.g., virus) to reduce the pathogen's impact on the individual and, if contagious, reduce the likelihood of spreading to others (e.g., reducing the “viral load” of potential super-spreaders to reduce risk of spreading to others). Preferably, the “load” is reduced by at least about 10% pathogens (e.g., viral particles, e.g., as measured by conventional swab testing), more preferably by at least about 20%, even more preferably by at least about 30%, even more preferably by at least about 50%, even more preferably by at least about 75%, even more preferably by at least about 90%, even more preferably by at least about 95% and most preferably by at least about 99%. Moreover preferably, the “load” is reduced by at least 10% pathogens (e.g., viral particles, e.g., as measured by conventional swab testing), more preferably by at least 20%, even more preferably by at least 30%, even more preferably by at least 50%, even more preferably by at least 75%, even more preferably by at least 90%, even more preferably by at least 95% and most preferably by at least 99%.

One aspect of the invention relates to methods of using particles optimized for therapeutic application to and within a host (e.g., human or patient), preferably for the therapeutic application to the upper respiratory system of a host (e.g., human or patient).

Preferably, the therapeutic compositions are both non-toxic and can be digested by humans or animals to allow for passage from the upper respiratory system to the digestive system or otherwise removed (e.g., cough, sneeze, dissolving) and also optimized to avoid and/or reduce risk of passage to the lower respiratory system. Preferably, the composition is also optimized to remain within upper respiratory system (e.g., nasal passages, throat) for a sufficient amount of time to provide effective treatment against the infection within the upper respiratory system and/or reduce the active viral or foreign substance load.

Preferably, the therapeutic compositions are not a pharmaceutical drug agent, more preferably do not include a pharmaceutical drug or agent and even more preferably do not contain a component that changes an organism's physiology or psychology when consumed or administered, nor otherwise intended to produce a biological effect other than optionally specifically binding to a cellular surface within the host during treatment (e.g., cellular surface within the upper respiratory system, local bacteria or pathogen of interest) and/or to reduce the infection, illness or disease, and/or one or more symptoms thereof by the elimination or reduction of the pathogen or allergen. Preferably, the therapeutic compositions target the pathogen (e.g., the pathogen's physiology, activity, etc.) by disrupting, destroying and/or inactivating the pathogen within the human or animal or plant and/or reduce the active viral or foreign substance load, preferably by either specifically binding to the pathogen and/or disrupting the outer membrane of the pathogen. Another advantage of the invention is the therapeutic compositions can be used with individuals not able or willing to use pharmaceutical drug and/or vaccines or the like. For example, according to one specific embodiment, the therapeutic compositions may only contain as “active agents” a plurality of inert, non-toxic microparticles having antibodies having binding specificity for the pathogen immobilized therefore and, optionally, one or more natural surfactants already typically present in the individual's lungs, such therapeutic compositions would likely be more tolerable to individuals unable and/or unwilling to be vaccinated and/or take other drugs.

Several of the embodiments of the invention include one or more particles. The administration of therapeutic compositions to humans, for example, and specifically to the upper respiratory system, according to these embodiments include considerations including how the composition is administered, how long a time is an effective amount of time to provide the desired result, while reducing risks to the body (e.g., lower respiratory system) and reducing any other risks to the human and/or animal.

For example, it is known that nano- or micro-particles can be hazardous to the respiratory system impacting a human's and/or animal's health. In humans, the respiratory system can be divided into two parts—the upper airway passages and the lower airway passages. The upper airway passages include the nose, nasal passages, mouth and the pharynx down to the vocal cords in the larynx. The lower airway passages start at the vocal cords, extend down the trachea (windpipe) and continue all the way down to the lung's small air sacs (alveoli) at the end of every branch of the bronchial tree. The bronchial tree includes the trachea, the bronchus (branches of the trachea going to each lobe of the lung), and bronchioles (branches of the bronchi).

The human throat (pharynx and larynx) is a ring-like muscular tube that acts as the passageway for air, food and liquid. It is located behind the nose and mouth and connects the mouth (oral cavity) and nose to the breathing passages (trachea [windpipe] and lungs) and the esophagus (eating tube). Tonsils are located at the back and sides of the mouth and adenoids are located behind the nose and both help to fight infections. The pharynx is the muscle-lined space that connects the nose and mouth to the larynx and esophagus (eating tube).

The pharynx allows both food and air to pass through separately, with food taking priority during eating. To prevent choking or breathing in of food/liquid particles, the area is structured so that air enters the larynx at the front; during eating, a small flap of connective tissue covers the airway and the food passes by that entrance, on to the esophagus. Because it is the top of the air passageway, the pharynx helps with speech as well. The pharynx has three physical sections: (i) nasopharynx (the upper portion of the back of the throat, from the line joining the soft/hard palate; down behind the back of the nasal area); (ii) otopharyx (the lower portion of the back throat; tonsils, soft palate and uvula form the back wall of otopharnx); and (iii) laryngopharynx (the area in the back of the throat corresponding to the cervical). The epiglottis is a flap of soft tissue and cartilage located just above the vocal cords. The epiglottis folds down over the vocal cords to help prevent food and irritants from entering the lungs. The esophagus refers to the thin muscular tube connecting the back of your throat to the opening of a human's stomach, through which food and drinks must pass in order to be fully digested and assimilated into the human's system.

The structure of the nose (bones and cartilage) causes inhaled air to swirl to deposit large particles, while the mouth has no filtering system. How far a particle moves into the air passages of the respiratory system depends on the size, shape, and density of the particulate material. In general, particles having an aerodynamic diameter of greater than 10 μm are deposited in the nasopharyngeal region (upper airway passages-nose, nasal cavity and throat). This mechanism is prominent because of the high air speed and the many turns in the nasopharyngeal air way. The changes in airflow direction cause many particles to hit the walls of the air passage and so the particles deposit or settle in this region.

According to preferred embodiments of the invention, the therapeutic compositions described herein, including compositions including particles, are configured and optimized to be administered to the upper respiratory area (e.g., nasal passages, throat), remain in the treatment area for an effective time period (e.g., preferably slow removal and reduced risk of triggering immediate cough/sneeze reflect), and then pass onto the esophagus and/or expelling (e.g., cough or sneeze) rather than entering the lower respiratory system.

Preferably, the therapeutic compositions described herein are configured and/or administered so that at least 90 wt % of the therapeutic composition passes onto the esophagus and/or are expelled (e.g., rinse/flush, cough) rather than entering the lower respiratory system, more preferably at least 95 wt % of the therapeutic composition, even more preferably at least 99 wt % of the therapeutic composition, even more preferably at least 99.9 wt % of the therapeutic composition, even more preferably all detectable amounts, and most preferred, 100 wt % of the therapeutic composition passes onto the esophagus and/or are expelled and/or preferably no detectible amount passes to the lower respiratory system.

Preferably, the therapeutic compositions described herein are configured and/or administered so that at least 90% (by particle count) of the therapeutic composition passes onto the esophagus and/or are expelled (e.g., rinse/flush, cough) rather than entering the lower respiratory system, more preferably at least 95% (particle count) of the therapeutic composition, even more preferably at least 99% (particle count) of the therapeutic composition, even more preferably at least 99.9% (particle count) of the therapeutic composition, even more preferably all detectable amounts, and most preferred, 100% (particle count) of the therapeutic composition passes onto the esophagus and/or are expelled and/or preferably no detectible amount passes to the lower respiratory system.

According to preferred embodiments, the composition is configured so that the amount particles that pass to the lower respiratory system are less than the amount that would negatively impact the patient (e.g., no measurable and/or detectable biological detrimental effect on the lower respiratory system or patient).

According to alternative preferred embodiments, the composition is configured to reduce the active viral load of the individual, preferably, reduce the number of active viral particles by 50% active viral particles, more preferably 75%, even more preferably by 90%, even more preferably by 95%, even more preferably by 99%, even more preferably by 99.9%, even more preferably by 99.99% and most preferred by 100%. According to one embodiment, the reduction in viral load is determined by conventional testing (e.g., swab test).

Individuals having a high viral load of Covid-19, for example, are believed to release a large amount of virus into the air resulting in increased detrimental spreading of the virus. (See, “Estimation of Aerosol Emissions from Simulated Individuals with Asymptomatic to Moderate COVID-19”, JAMA Network Open, Jul. 27, 2020). Accordingly, reducing the active viral load within a patient can not only reduce the risks for the patient, but also reduce the risk of the patient spreading the virus to others.

According to preferred embodiments, the least one particle is substantially non-respirable, even more preferably the least one particle is a non-respirable particle. Preferably, the shape, size, surface characteristics and/or composition of the particle is configured to prevent the particle(s) from entering the lower respiratory system. Preferably, the composition comprises a plurality of particles and at least 99.99% of the particles are non-respirable particles and/or less than 0.01% of the particles are respirable particles, more preferably less than 0.001% of the particles are respirable particles, even more preferably less than 0.0001% of the particles are respirable particles, even more preferably less than 0.00001% of the particles are respirable particles and more preferred no measurable and/or detectable amounts of the particles are respirable particles.

Moreover, preferably, the particles are configured (e.g., shape, size, composition, surface, etc.) to reduce or at least slow the particles being washed out and/or expelled by the normal mucociliary clearance mechanisms to increase the effective amount of the treatment and/or reduce need for frequent reapplications.

Preferably, the selection of the particle type and particle characteristics is balanced between avoiding being too small (which increases risk of migration to lower respiratory system) and avoiding being too large to result in discomfort within treatment area, triggering premature cough/sneezing and/or too quick of a removal by the normal mucociliary clearance mechanisms. That is, the particles are optimized (by size, composition, surface morphologies, etc.) to increase the effective amount of treatment, while reducing the risk to the individual being treated.

For example, as in the case of nasal breathing, virtually no particles above 10 μm descend to the lower respiratory system and particle deposition typically increases with particle size. Particles of irregular shape often behave in unpredictable ways and therefore are preferably avoided unless the irregular shape is optimized for treatment (discussed below in certain embodiments). For example, asbestos fibers up to 200 μm in length but only 0.5 μm in diameter may be found in peripheral airways and lung parenchyma as may be some smaller rod-shaped or curvilinear spores. In these cases, the particles have essentially been propelled down the airways like an equivalent diameter spear aligned with the air flow facilitating migration deeper into the distal airways of lungs. Accordingly, elongate spear-like particles are preferably avoided to reduce risk to lower respiratory system.

A healthy tracheobronchial tree necessitates efficient clearance of pollens, pollutants, infectious agents, and other cellular debris. Efficient clearance is accomplished by the process of mucociliary action, cough, and alveolar clearance mechanisms. Most pollen grains, which are 10 to 20 μm, are largely deposited in the nose, whereas many smaller particles in the range of 1 to 5 μm, such as mold spores, frequently reach the distal airways. Particles deposited on the nasal mucosa are typically rapidly transported to the pharynx by mucociliary transport within 15 to 30 minutes of impaction and are subsequently swallowed.

Mucociliary clearance involves the protective mechanisms afforded by mucus, its cellular contents, and its local content of antiproteinases, lysozyme, and other antimicrobial substances. Ciliated cells line the airways interspersed among goblet cells from the anterior nasopharynx to the terminal bronchi. Beating cilia penetrate the mucous layer in groups of cilia and beat in a synchronized coordinated fashion in an overall cephalad direction. The rapid movement of cilia creates a series of waves that, in a continuous and synchronized manner, propel the mucus, exfoliated cells, and entrapped particles (e.g., pollen, dust, etc.) out of the respiratory tract to the pharynx. The mucus is finally swallowed or, when present in large amounts, is coughed up out of the conducting system. Cilia movements have been compared with those of an escalator with progressively increasing speeds from the terminal bronchiole to the trachea. Most inhaled local debris of relatively large size plus other comparatively insoluble particles will be transported in a cephalad direction up this escalator mechanism by the mucous layer to the larynx and ultimately swallowed or cleared by way of the coughing mechanism. Mucus acts primarily as a barrier and a vehicle, and it is a complex mixture of water, glycoproteins, immunoglobulins, lipids, and electrolytes. These substances are produced by goblet (mucous) cells, serous cells, submucosal glands, and fluid from transepithelial ion and water transport. Once serous fluid and mucus are secreted onto the surface of the respiratory mucosa, a thin, double-layer film of mucus is formed on top of the cells. The outer layer of this film is in a viscous gel phase, whereas the inner layer, which is in a fluid or sol phase, is directly in contact with cilia. Healthy airways usually clear such particulates within a matter of only 6 hours, and the entire “escalator” can be cleared within a 24-hour period in a healthy person (see, e.g., Inhaled Particles and Respiratory Disease, by Salvaggio, The Journal of Allergy and Clinical Immunology, Vol. 94, Issue 2, pp. 304-309, Aug. 1, 1994, hereby incorporated by reference in its entirety).

The swallowing mechanism is done in three stages: the oral or voluntary stage, the pharyngeal stage and the oesophageal stage. In the oral stage the tongue is lifted to propel the bolus of food into the pharynx. The bolus stimulates tactile receptors of the pharynx to initiate the swallowing reflex. In the pharyngeal stage, the soft palate is pulled upwards and approximated against the pharynx where the palatopharyngeal and palatoglossal folds move inwards towards one another, preventing reflux of food into the nasopharynx. The larynx moves upwards and is approximated against the epiglottis. Food is thus prevented from entering the trachea. The upper oesophageal sphincter (the cricopharyngeal part of the inferior constrictor) relaxes and the superior constrictor of the pharynx contracts to force the bolus onwards. The bolus is then propelled onwards by sequential contraction of the superior, middle and inferior constrictors of the pharynx. This produces a peristaltic wave pushing the bolus towards the upper end of the oesophagus. During the pharyngeal stage respiration is reflexively inhibited. After the bolus has passed the upper oesophageal sphincter reflexively constricts. The bolus is propelled downwards by the primary peristaltic wave caused by impulses originating in the swallowing centre in the medulla and conducted via the Xth nerve to the myenteric plexus of the oesophagus.

The cough reflex protects the airways and lungs from aspiration, inhaled irritants, particulates and pathogens and clears the air spaces of accumulated secretions.

According to preferred embodiments of the invention, the therapeutic composition is configured to reduce the cough reflex when the composition administration is to the throat (or the particles migrate from the nasal passages to the throat after nasal administration). According to preferred embodiments, the particles are optimized to reduce and/or delay, more preferably eliminate the cough reflex. According to other preferred embodiments, numbing agents and/or cough suppressants are incorporated in the therapeutic composition before oral and/or nasal administration (e.g., incorporated into the therapeutic composition) and/or administered to the individual prior to and/or after administration of the therapeutic composition.

Sneezing is also a protective reflex and is most commonly caused by the irritation of the nasal mucosa. The irritant might be an unusual smell, dust, animal dander, pepper, viruses that attack the mucous membranes or a variety of other substances. According to preferred embodiments of the invention, the therapeutic composition is configured to reduce and/or delay and/or eliminate the sneeze reflex when the composition administration is nasally (e.g., nasal spray). According to preferred embodiments, the particles are optimized to reduce, more preferably eliminate a sneeze reflex. For example, preferably therapeutic compositions are applied via a liquid spray to form a liquid film within the nasal passages with any particles dispersed in the film. According to other preferred embodiments, numbing agents and/or sneeze suppressant agents are incorporated in the therapeutic composition and/or administered to the individual prior to and/or after administration of the therapeutic composition.

One of ordinary skill in the art would understand the therapeutic compositions described herein can be adapted or configured by optimizing the amount administered and the manner of administration, along with optimizing the composition, in particular the particles used, in view of the area of anatomy of treatment and how that anatomy reacts to any particles including optimized treatment time in the target area of interest.

One aspect of the invention relates to a therapeutic composition for treatment of an illness and/symptoms caused by a pathogen, allergen or other substance, the composition comprising at least one particle and at least one antibody structure comprising an antibody or antibody fragment that specifically binds to the pathogen, allergen or other substance, wherein the at least one antibody structure is attached to, connected to, adsorbed onto, absorbed into, or embedded in the particle, wherein the particle has a diameter (preferably an aerodynamic diameter) of greater than 10 μm and less than 100 mm (preferably less than 200 μm) and/or the therapeutic composition is essentially nontoxic to human, animal and/or plant life.

According to alternative embodiments, the therapeutic composition comprises allergen-specific antibodies to specifically bind to allergens (e.g., pollen, dust mites, dust mite parts and/or excretions, pet dander, etc.). Preferably, the composition is configured to be applied to the upper respiratory system and comprises microparticles having allergen-specific antibodies (e.g., IgE antibodies) attached thereto and configured to specifically bind the allergen and subsequently removed from the treatment area (e.g., applying flushing solution, normal mucociliary clearance mechanisms, migrating to the digestive tract, cough/sneeze, dissolving). (See, for example, Allergen-Specific Antibodies Regulate Secondary Allergen-Specific Immune Responses, by Eckl-Doma et al., Frontiers in Immunology, 2019 Jan. 17 (PMID: 30705676), hereby incorporated by reference in its entirety).

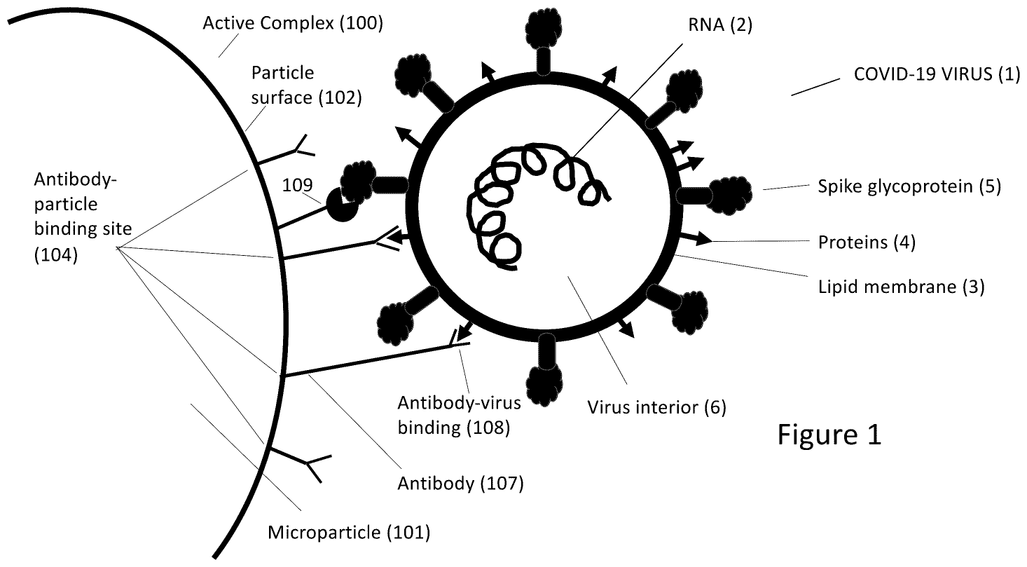

FIG. 1 is a graphical representation of an active complex 100 according to one embodiment of the invention. The active complex 100 comprises microparticle 101 having particle surface 102. Active complex 100 further includes antibodies 107 attached to particle surface 102 at antibody-particle binding sites 104. Methods of attaching antibodies 107 to particle surface 102 are well known in the art (including the descriptions set forth and/or incorporated by reference herein). Antibodies 107 include one or more antibodies that specifically bind to one or more binding sites on the target pathogen, shown as a virus 1 (COVID-19 virus).

As shown in FIG. 1, virus 1 includes a lipid membrane 3 including proteins 4 and spike glycoproteins 5 around the outer lipid membrane 3 of virus 1. Membrane 3 encloses a virus interior 6 which encompasses RNA 2.

Virus 1 is shown captured by active complex 100 by antibodies 107 capable of specifically binding to one or more proteins 4 and/or antibodies 109 capable of specifically binding to one or more glycoproteins 5. Antibodies 107 can include different antibodies (e.g., antibodies 109) that specifically bind to different components of the virus (e.g., different proteins 4 and/or glycoproteins 5). According to preferred embodiments, the antibodies specific bind to spiked glycoproteins, preferably on the surface of the virus membrane as shown in FIG. 1.

Preferred embodiments relate to therapeutic compositions comprising a plurality of active complexes as shown in FIG. 1.

According to preferred embodiments, antibodies 107 include at least 100 antibodies per particles, preferably at least 1000, more preferably at least 10,000, even more preferably at least 100,000 antibodies per particle, even more preferably at least 1,000,000 antibodies per particle, even more preferably at least 10,000,000 antibodies per particle, even more preferably at least 100,000,000 antibodies per particle.

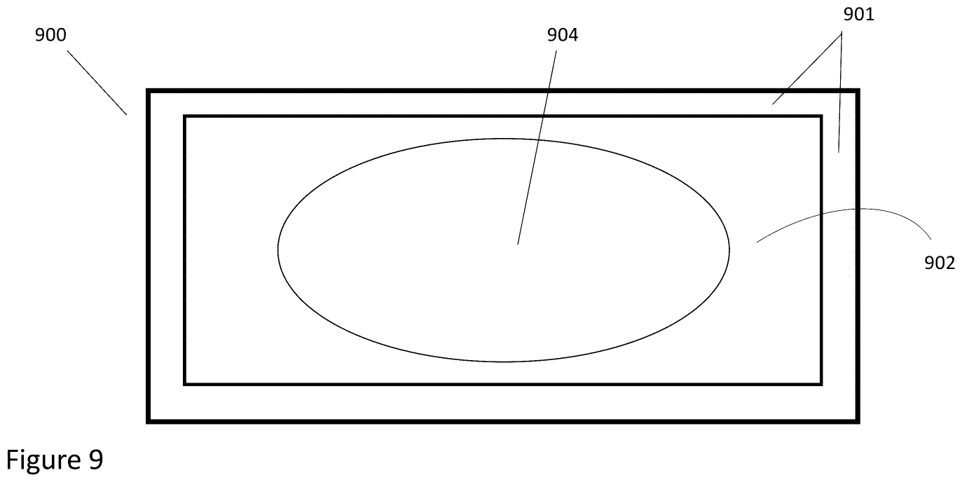

According to preferred embodiments, the density of a monolayer of antibodies immobilized on the surface of the particle or support or wound dressing (e.g., FIG. 9), etc. is between 100 and 200,000 per micrometer squared, more preferably between 1,000 and 100,000 per micrometer squared, even more preferably between 5,000 and 50,000 per micrometer squared, even preferably between 10,000 and 30,000 per micrometer squared, even preferably between 15,000 and 25,000 per micrometer squared and most preferred approximately 20,000 per micrometer squared.

The effectiveness of the therapeutic composition will generally be dependent on immobilization of the capture antibodies onto a solid support with a sufficient surface density, a conformation that is representative of their native, solution-phase state, and an orientation that maximizes their antigen capture potential. The antibodies can be immobilized on the particle or support (or on structures such as wound dressings as described below) using known methods including surface modification and design (with sections on surface treatments, three-dimensional substrates, self-assembled monolayers, and molecular imprinting), covalent attachment (including targeting amine, carboxyl, thiol and carbohydrates, as well as “click” chemistries), and (bio) affinity techniques (with sections on material binding peptides, biotin-streptavidin interaction, DNA directed immobilization, Protein A and G, Fc binding peptides, aptamers, and metal affinity). (See, for example, Orientation and Characterization of Immobilized Antibodies for Improved Immunoassay (Review) by Welch, Biointerphases, Vol. 12, Issue 2 (16 Mar. 2017), which describes various methods and process for immobilizing antibodies and hereby incorporated by reference in its entirely).

According to one embodiment, the particles or support are plasma treated prior to immobilizing the antibodies (see, e.g., A. Hasan and L. M. Pandey, Polym.-Plast. Technol. Eng. 54, 1358 (2015), hereby incorporated by reference in its entirely).

According to another embodiment, a self-assembled monolayer (SAM) is formed including molecules providing functional groups for subsequent covalent attachment of the antibodies (See, e.g., S. K. Vashist, E. Lam, S. Hrapovic, K. B. Male, and J. H. Luong, Chem. Rev. 114, 11083 (2014), hereby incorporated by reference in its entirely).

According to another embodiment, the antibodies are molecular imprinted, preferably using a molecular template to product target-specific binding regions on the surface. (See, e.g., N. Bereli, G. Erturk, M. A. Tumer, R. Say, and A. Denizli, Biomed. Chromatogr. 27, 599 (2013); A. Bossi, S. A. Piletsky, E. V. Piletska, P. G. Righetti, and A. P. Turner, Anal. Chem. 73, 5281 (2001); M. Cretich, F. Damin, G. Pirri, and M. Chiari, Biomol. Eng. 23, 77 (2006), each hereby incorporated by reference in its entirely).

According to another embodiment, the antibodies are immobilized using covalent coupling, preferably using amine and/or carboxyl groups (e.g., EDC/NHS coupling) or thiol groups or “click” chemistry, or carbohydrate groups. (see, e.g., K. Trilling, T. Hesselink, A. van Houwelingen, J. H. Cordewener, M. A. Jongsma, S. Schoffelen, J. C. van Hest, H. Zuilhof, and J. Beekwilder, Biosens. Bioelectron. 60, 130 (2014); A. Béduneau, P. Saulnier, F. Hindré, A. Clavreul, J. C. Leroux, and J. P. Benoit, Biomaterials 28, 4978 (2007); E. Mauriz, M. C. Garcia-Fernandez, and L. M. Lechuga, Trac-Trends Anal. Chem. 79, 191 (2016); S. D. Carrigan, G. Scott, and M. Tabrizian, Langmuir 21, 5966 (2005), each hereby incorporated by reference in its entirely).

According to another embodiment, the antibodies are immobilized using affinity immobilization techniques including (i) material binding peptides; (ii) biotin-streptavidin; (iii) DNA directed immobilization; (iv) protein A and protein G (or other small proteins, preferably derived from bacteria, which can specifically bind the Fc portion of antibodies); (v) Fc-binding peptides and aptamers; (vi) nucleotide binding site; and/or (vii) metal affinity. (See, e.g., Y. Jung, H. J. Kang, J. M. Lee, S. O. Jung, W. S. Yun, S. J. Chung, and B. H. Chung, Anal. Biochem. 374, 99 (2008); J. E. Hale, Anal. Biochem. 231, 46 (1995); N. J. Alves, T. Kiziltepe, and B. Bilgicer, Langmuir 28, 9640 (2012); L. Bjorck and G. Kronvall, J. Immunol. 133, 969 (1984).94. P. L. Ey, S. J. Prowse, and C. R. Jenkin, Immunochemistry 15, 429 (1978); E. Seymour, G. G. Daaboul, X. Zhang, S. M. Scherr, N. L. Unlu, J. H. Connor, and M. S. Unlu, Anal. Chem. 87, 10505 (2015); I. H. Cho, J. W. Park, T. G. Lee, H. Lee, and S. H. Paek, Analyst 136, 1412 (2011); K. Hernandez and R. Fernandez-Lafuente, Enzyme Microb. Technol. 48, 107 (2011); and A. Makaraviciute and A. Ramanaviciene, Biosens. Bioelectron. 50, 460 (2013), each hereby incorporated by reference in its entirely).

Preferably, the antibodies and/or other active agent cover substantially the entire surface of the particle (preferably with spacing between antibodies) and/or are distributed throughout the entire surface and/or distributed throughout an entire active surface of the particle (e.g., concave portion, exposed side of particle, etc.) and/or immobilized with pores, cavities or other void space(s) within the particle).

Preferably, the particle has a largest dimension greater than 1 μm and less than 100 mm, more preferably 5 μm and less than 75 mm, even more preferably 10 μm and less than 50 mm and most preferred between 10 μm and 200 μm.

According to further preferred embodiments, the particle has a largest dimension between 10 μm and 200 μm, more preferably between 20 μm and 100 μm.

According to alternative preferred embodiments, the therapeutic composition comprises particles, and the particle maximum distribution is bimodally or trimodally or multimodally distributed. For example, a plurality of first microparticles having largest dimensions of approximately 20 μm and a plurality of first microparticles having largest dimensions of approximately 100 μm. Therapeutic composition comprising particles with such particle size distributions (i) improves particle packing storage, (ii) can increase distribution of the particles when administered to the upper respiratory system as a result of the varied morphologies on the interior surfaces of the upper respiratory system; (iii) varies the treatment time when dissolvable particles are used since smaller particles will dissolve more quickly compared to larger particles; and/or (iv) combinations thereof.

According to alternative preferred embodiments, the therapeutic composition comprises two or more different types of particles (e.g., dissolvable particles and digestible particles).

Preferably, the therapeutic composition is nontoxic to human, animal and/or plant life in the effective amounts used for treatment and/or approximate range of effective amounts used for treatment.

Preferably, the therapeutic composition is an environmentally compatible and/or biocompatible composition.

Preferably, the therapeutic composition is digestible by humans in the effective amounts used for treatment and/or approximate range of effective amounts used for treatment.

Preferably, the therapeutic composition operates by linking the pathogen to the particle via the antibody within the upper respiratory system (e.g., nasal passages, throat) and preferably passing the particle-antibody-pathogen complex into the digestive tract and/or by expelling the composition (e.g., eventually triggering a cough/sneeze to expel the complex from the upper respiratory system through mouth/nose, flushing out with a rinse (e.g., saline rinse)). Alternatively, or additionally, the therapeutic composition operates by otherwise inactivating the pathogen.

Preferably, the pathogen is a virus or a bacteria.

Preferably, the at least one particle has an outer surface having a plurality of said antibody structures attached to, connected to, adsorbed onto, absorbed into, or embedded in the particle and/or the outer surface.

Preferably, the at least one particle comprises two or more different antibodies, each having different binding specificities for the pathogen (or allergen or other foreign substance). For example, a first antibody specific for a first protein in the virus membrane and a second antibody specific for a second protein in the virus membrane. According to preferred embodiments, the at least one particle has an outer surface having a plurality of the first antibody structures and a plurality of the second antibody structures. According to preferred embodiments, the particle comprises a first antibody, a second antibody and a third antibody for the pathogen (i.e., multiple different “pathogen antibodies”).

Preferably, the composition comprises a plurality of particles, each having a plurality of the antibody structures (e.g., attached, linked and/or or connected to the outer surface of each particle).

Preferably, the at least one particle comprises a plurality of the at least one antibody structure attached to, connected to, adsorbed onto, absorbed into, or embedded in the particle.

Preferably the therapeutic composition comprises a plurality of the at least one particle. Preferably, the effective amount of therapeutic composition, including prophylactically effective amount, for treatment includes at least 10 particles, more preferably at least 50 particles, even more preferably at least 100 particles, even more preferably at least 500 particles, even more preferably at least 1000 particles, even more preferably at least 5000 particles, even more preferably at least 10,000 particles, even more preferably at least 100,000 particles, even more preferably at least 1,000,000 particles and even more preferably at least 100,000,000 particles.

According to one embodiment, the therapeutic composition further comprises at least one second antibody or second antibody fragment specific to one or more cells that line the nasal passages, oral cavity and/or throat and/or one or more bacteria that line the nasal passages (“local bacteria”), oral cavity and/or throat and the at least one second antibody or second antibody fragment is attached or connected to the particle. That is, at least one second antibody or second antibody fragment to link, bind, adhere or otherwise attach the particles to the interior surfaces of the throat and/or nasal passages and/or other location (herein “anchor antibody”). Preferably the at least one second antibody or second antibody fragment is configured to increase the effective treatment time period within the upper respiratory system by at least temporarily attaching the active particle to the interior surfaces of the throat and/or nasal passages and/or other treatment location.

According to one preferred embodiment, the at least one second antibody or second antibody fragment is specific for binding to ciliated cells in the superficial epithelium, and, more preferably, not to the nasal submucosal glands.

According to another preferred embodiment, the at least one second antibody or second antibody fragment is specific for binding to ciliated cells and/or type-2 pneumocyte cells.

According to another preferred embodiment, the at least one second antibody or second antibody fragment is specific for binding to cells expressing human angiotensin-converting enzyme (ACE2).

According to another further preferred embodiment, the at least one second antibody or second antibody fragment is specific for binding to ciliated cells, preferably in the superficial epithelium, within the upper respiratory system, more preferably within the oropharynx.

According to another further preferred embodiment, the at least one second antibody or second antibody fragment is specific for binding to ciliated cells, preferably in the superficial epithelium, within the nasal system.

Preferably, each of said at least one particle has an outer surface and the therapeutic composition further comprises the at least one anchor antibody structure including an anchor antibody or anchor antibody fragment specific to one or more cells that line the nasal passages, oral cavity and/or throat and/or one or more local bacteria and said at least one antibody structure is attached or connected to the outer surface of the particle. Alternatively, the therapeutic composition comprises a plurality of particles having at least one anchor antibody and a plurality of different particles not having at least one anchor antibody, preferably wherein the different particles are configured to link to the other particles (e.g., aggregate or agglomerate via adhesives, ligands, etc.).

Preferably, one or more of the particles comprise a “bifunctional linking reagent” or “bifunctional linkers” to bind to the local bacteria, cells, and/or other particles.

According to preferred embodiments, the one or more of the particles comprise at least one bacterial-specific liquid specific to one or more cells and/or one or more local bacteria.

According to another embodiment, each of said at least one particle has an outer surface and the therapeutic composition further comprises at least one second (or anchor) antibody structure including a second antibody or second antibody fragment specific to one or more cells and/or one or more local bacteria and said at least one second antibody structure is attached or connected to the outer surface of the particle.

According to another embodiment, each of said at least one particle has an outer surface having a first side and an opposing second side and wherein the therapeutic composition further comprises at least one anchor antibody structure attached or connected to the first side of the outer surface of the particle and the at least one pathogen antibody structure attached or connected to the second side of the particle. Preferably, the at least one pathogen antibody structure has a binding affinity for the pathogen (e.g., virus) greater than the binding affinity of the at least anchor antibody to the one or more cells and/or local bacteria (e.g., first antibody has an approximate K_{D} value greater than about 10⁻⁹ while the anchor antibody has an approximate K_{D} value less than about 10⁻⁹, preferably the first antibody has an approximate K_{D} value greater than about 10⁻¹⁰ while the anchor antibody has an approximate K_{D} value less than about 10⁻⁹ or the first antibody has an approximate K_{D} value greater than about 10⁻⁹ while the anchor antibody has an approximate K_{D} value less than about 10⁻⁸) (e.g., as measured by the OI-RD method described at abcam dot com). Specifically, most antibodies have K_{D} values in the low micromolar (10⁻⁶) to nanomolar (10⁻⁷ to 10⁻⁹) range. High affinity antibodies generally considered to be in the low nanomolar range (10⁻⁹) with very high affinity antibodies being in the picomolar (10⁻¹²) range. Preferably, according to these embodiments, the pathogen (e.g., virus) will link more strongly to the antibody structure (and thus the particle) compared to how strongly the anchor antibody will link to the to the one or more cells and/or local bacteria thus allowing the complex (particle-antibody structure-pathogen) to detach or “fall off” from the one or more cells and/or local bacteria to eventually pass out of the upper respiratory system (e.g., to digestive system or ejected by flushing, cough or sneeze).

According to alternative embodiments, the at least one antibody structure has a binding affinity for the virus (or other foreign substance) less than the binding affinity of the at least one anchor antibody. According to these embodiments, the active components of the therapeutic composition (antibody structure and particle) are configured to remain active in the area of treatment for longer if the anchor antibody affinity to the one or more cells and/or local bacteria is sufficiently strong to slow removal of particle from treatment area.

Another embodiment of the invention relates to the use of one or more adhesives (alone or in addition to anchor antibodies described above) to promote adherence of the particle complex to the surface of treatment (e.g., to the throat or nasal passages). Preferably, the therapeutic formulation further comprises one or more mucosa-adhesives (e.g., such as the adhesives described in Korean Patent Publication KR100413877B1, U.S. Pat. No. 6,319,513 to Dobrozsi, U.S. Pat. No. 5,047,244 to Sanvordeker et al. and U.S. Pat. No. 5,554,380 to Cuca et al., each hereby incorporated by reference in its entirety) to allow the particles to remain at the treatment location for an longer period of time to act on viruses within the upper respiratory system (e.g., nasal passages, mouth and/or throat). For example, ARcare® mucosal adhesive systems such as sublingual and mucosal systems for adhesion to mucosal tissues for extended periods of time. Preferably, the adhesives used are formulated for removal or dissolving or absorption upon end of treatment, or to erode at a specific or estimated wear or usage time.

Preferably, the at least one particle has an outer surface including one or more adhesives configured and/or adapted to adhere or increase adherence to one or more cells that line the nasal passages, oral cavity and/or throat and/or one or more local bacteria that line the nasal passages, oral cavity and/or throat.

Preferably, each of said at least one particle has an outer surface having a first side and an opposing second side and wherein the first side includes one or more adhesives configured and/or adapted to adhere or increase adherence to one or more cells that line the nasal passages, oral cavity and/or throat and/or one or more local bacteria that line the nasal passages, oral cavity and/or throat and said at least one antibody structure is attached or connected to the second side of the outer surface of the particle.

Preferred embodiments of the invention include particles having one or more membranes on the outer surface in addition to the above-described pathogen antibody, anchor antibody and/or adhesives. Preferably, the particle further comprises a membrane, wherein the membrane encapsulates the particle and wherein the membrane is a naturally occurring cellular membrane, a pathogen membrane, or a synthetic membrane, such as the membranes described in WO2013056132/US20170232115 to Ashley; EP 3412282/US20130337066 to Zhang, each describing methods of manufacturing nanoparticles having membrane layers for use in drug delivery and hereby incorporated by reference. Preferably, the membrane is optimized to bind to the pathogen of interest (e.g., virus, bacteria, etc.). Preferably, the membrane is optimized to attract and/or bind and/or link the pathogen of interest (e.g., virus, bacteria, etc.) to the particle to bind with the pathogen antibody.

One preferred embodiment of the invention relates to a therapeutic composition as described herein dispersed in a liquid, wherein liquid formulation is configured for administering to a subject topically, by injection, to the eye, orally, by inhalation, dermally (e.g., patch), sublingually, suppository, or otherwise administered, preferably configured for administering to apply the composition to the throat, nasal passages and/or eyes.

According to preferred embodiments, the particles include linking antibodies, linkages and/or surface properties optimized to result in the agglutination and/or aggregation of particles within the upper respiratory system to form larger particle assemblies to trigger cough/sneeze and/or reduce likelihood will pass to lower respiratory system. Preferably, each particle comprises one or more ligands configured to connect or link or bind to other particles to result in aggregates/assemblies and/or the particles have surface characteristics to result in agglutination and/or aggregation and/or assembling to result in larger particles over a period of time to generate cough/sneeze and/or otherwise facilitate removal by biological mechanisms, flushing with saline solutions, or the like. Preferably, one or more particles include one or more functional linking reagents and/or bifunctional linkers, preferably configured to link the particles to other particles.

Preferably, the therapeutic composition (including the liquid containing the components) to be administered is initially configured and/or adapted to reduce agglutination and/or aggregation of particles so that the particles are dispersed upon administration to the area of treatment. Proteins are well known to improve dispersion of nanoparticles by forming particle-protein corona, thereby diminishing particle-particle interactions. For example, albumin or globulin proteins have been widely employed to improve dispersion of nanoparticles (Wang J., Jensen U. B., Jensen G. V., Shipovskov S., Balakrishnan V. S., Otzen D., Pedersen J. S., Besenbacher F., Sutherland D. S. Soft interactions at nanoparticles alter protein function and conformation in a size dependent manner. Nano Lett. (2011); 11:4985-4991. doi: 10.1021/n1202940k, hereby incorporated by reference in its entirety). For example, it is known that 0.01% Tween 80 has little effect on most physiological functions of any cells or tissues, thus being useful as a biocompatible vehicle for particle dispersing for other applications.

Preferably, the therapeutic composition for administration comprises a dispersant (e.g., one or more surfactants such Tween 20, Tween 80, or the like, preferably in low levels (e.g., preferably less than 1 wt %, more preferably less than 0.1 wt %, even more preferably less than 0.05 wt %, and most preferred about 0.01 wt %)).

Preferably, the composition is configured so that, after application, the resultant movement of the particles within the upper respiratory system will result in the particles connecting resulting in the agglutination and/or aggregation of particles. That is, while in the container (i.e., prior to administration) or during spraying, the composition is configured to reduce the tendency of the particles to aggregate, while preferably, after administration to the treatment surface, the composition after application is adapted or configured to allow the particles to aggregate, preferably over a period of time, to facilitate eventual removal from treatment area. For example, the composition may include dispersants that function in the container but not after application.

Another embodiment of the invention relates to therapeutic composition for treatment of an illness caused by a virus (or other pathogen or allergen or substance), the composition comprising at least one antibody support and at least one antibody structure comprising an antibody or antibody fragment that specifically binds to the virus, wherein the at least one antibody structure is attached to, connected to or embedded in the at least one antibody support, wherein the at least one antibody support has an aerodynamic diameter of greater than 10 μm and less than 100 mm (preferably greater than 20 μm and less than 200 μm) and the therapeutic composition is an environmentally compatible and/or biocompatible composition essentially nontoxic to human, animal and/or plant life. Specifically, instead of limited to use of a “particle” as described above, the therapeutic composition may use a particle (as described above or otherwise herein) or supramolecules (e.g., such as those described in U.S. Pat. No. 6,814,964 to Virtanen et al, hereby incorporated by reference in its entirety) or other microstructures including naturally occurring microstructures (e.g., pollen, inactivated viruses, etc.) or film-forming compositions or pastes.

According to preferred embodiments, the at least one antibody support (or at least one microplatform) has a plate-like or tube-like shape (e.g., a short tube with ratio of length to diameter less than 20, preferably less than 10, and more preferably less than 5), preferably the at least one antibody support (or at least one micro-platform) is plate-like to increase the surface area. Alternatively, the at least one antibody support (or at least one micro-platform) has a cylindrical or spherical shape.

According to one embodiment, the at least one antibody support (or at least one micro-platform) has spherical shape (e.g., zeolite, carbon buckyball, molecular sieve, microporous material, etc.) having pores on the outer surface, wherein the at least one antibody structure (and/or active agent) are immobilized within the pores and/or other voids.

According to alternative embodiments, the antibody support (or microplatform) is a biological material (e.g., pollen or pollen fragment (preferably sized 10-40 microns)), edible nanoparticle (e.g., plant-derived edible nanoparticle, preferably nanosized membrane vesicles or fragments derived from edible plants)).

According to alternative embodiments, the therapeutic compositions use edible (capable of being digested) and/or biocompatible particles capable of being injected into a host (e.g., bloodstream for treatment) and/or allowed into the lower respiratory system without risks of damaging inflammation and/or capable of being absorbed and/or removed by the body's mechanisms for removing foreign particulates. Preferably, the particles further comprise a coating to slow the particle dissolving or being absorbed or removed.

According to certain preferred embodiments of the invention, the therapeutic compositions are configured to destroy or disrupt the membrane of the pathogen and/or otherwise inactivate or kill the pathogen (and/or other foreign substance).

Another aspect of the invention relates to particles configured to destroy or disrupt the membrane of the pathogen and/or otherwise inactivate or kill the pathogen via mechanical movements and/or flexing of particle to tear or otherwise destroy or disrupt the membrane. According to this alternative embodiment, the antibody support is flexible (e.g., can bend and/or stretch and/or twist and/or expand/contract) and changes shape and/or size sufficient to distort the attached pathogen membrane so that the membrane ruptures, tears, breaks or otherwise makes the pathogen inactive. For example, a virus is attached to the support via one or more antibodies and the flexing of the support (e.g., by movement of the support within the throat, muscle movement, swallowing motion, etc.) causes the virus membrane to tear or rupture as the antibody support flexes, twists or otherwise changes shape and/or size (e.g., by absorbing water, movement along the cilia, etc.). Specifically, for example, while the particles are attached or adhering to the throat, the mucociliary clearance mechanisms and/or cilia movements will move and/or change the particle's orientation and/or location and will cause the particle to flex, twist and/or alter shape and/or size when optimized to be flexible resulting in disruption to the pathogen(s) attached thereto. Preferably, the particle or support is thin (e.g., less then 2 microns) relative to the width or length of the particle (e.g., greater than 10 microns) such as a thin plate-like structure that is capable of flexing when moved by the cilia, for example.

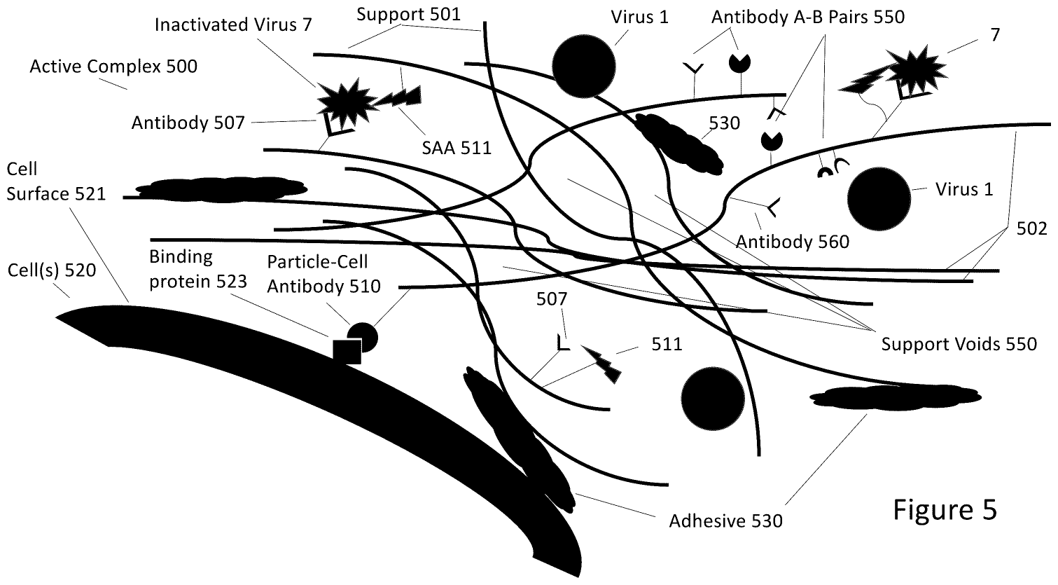

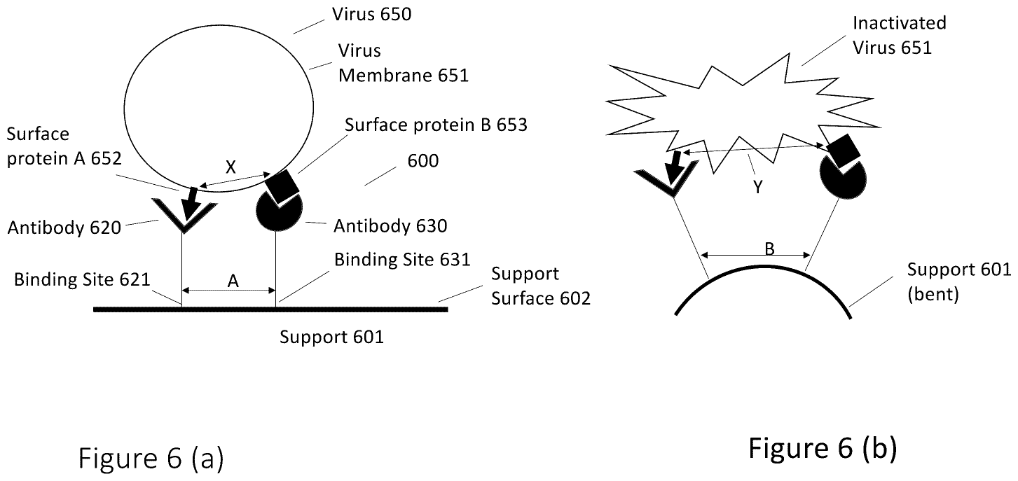

FIG. 6(a) is a graphical representation of another embodiment of the invention showing active complex 600 including support 601 having support surface 602. Active complex 600 further comprises antibody 620 linked to support surface 602 at binding site 621 and antibody 630 linked to support surface 602 at binding site 631, forming an “antibody pair”. Preferably, antibody 620 is adjacent antibody 630 on support surface 602, as shown in FIG. 6(a). Alternatively, each “antibody pair” can be located separately as shown in FIG. 5 and configured so that each antibody within the antibody pair can bind to the same virus particle membrane.

Also shown is virus 650 including virus membrane 651 and surface protein A 652 and surface protein B 653 on the surface of virus membrane 651. Antibody 620 is specifically bound to surface protein A 652 and antibody 630 is specifically bound to surface protein B 653.

Distance A as shown as arrows is the distance between antibody 620 and antibody 630 (e.g., distance between binding site 621 and binding site 631). Distance X is the distance between a point on the surface of virus membrane 651 (e.g., surface protein A 652) and another point on the surface of virus membrane 651 (surface protein B 653). In FIG. 6(a) the distances A and X are shown while support 601 is “at rest” or not bent or otherwise misshapen.

FIG. 6(b) shows active complex 600 and virus 650 of FIG. 6(a) after support 601 has been flexed or bent, as shown. Distance B corresponds to distance A of FIG. 6(a) and is larger than distance A after the support 601 is bent, as shown. Similarly, distance Y is larger than distance X resulting in the tearing or otherwise destroying virus 650 as shown as inactivated virus 651. That is, as support 601 is bent, antibody 620 and antibody 630 are separated which, in turn, separates surface protein A 652 and surface protein B 653 sufficiently to tear, rupture or otherwise disrupt the virus membrane 651 of virus 650 forming inactivated virus 651.

According to preferred embodiments, distance B is at least 5% larger than distance A, preferably at least 10% larger, more preferably at least 15%, more preferably at least 20%, more preferably at least 25% and most preferred at least 30%.

According to preferred embodiments, distance Y is at least 5% larger than distance X, preferably at least 10% larger, more preferably at least 15% and most preferred at least 20%.

Preferably, distances B and/or Y are sufficiently larger than corresponding distances A/X to effectively inactivate the virus.

According to one embodiment, support surface 602 comprises not only antibody 620 and antibody 630 but also one or more, preferably two or more, even more preferably three or more and most preferably four or more different antibodies for specifically binding different components of the virus.

According to preferred embodiments, the “different” antibodies are specific for the same protein or other moiety of the virus as the virus may have multiple copies of the protein or other moiety on the surface of the virus (e.g., multiple “copies” of the same antibody are included on the particle, for example, surface protein A 652 and surface protein B 653 are the same). According to alternative embodiments, different antibodies that are specific to different proteins or other moieties are used (e.g., surface protein A 652 and surface protein B 653 are different proteins).

According to another embodiment, the different antibodies (e.g., antibody 620 and antibody 630) are applied to support 602 in a pattern, preferably an alternating pattern.

According to an alternative embodiment, the antibodies are applied randomly and in sufficient numbers to result to different antibodies binding to a single virus membrane.

According to another embodiment, the antibodies are applied in a pattern and in sufficient numbers to result to different antibodies binding to a single virus membrane.

Preferably, the length of the linkages attached the antibodies is bi-modally or tri-modally distributed and/or otherwise varied to optimize the antibodies binding to pathogen(s).

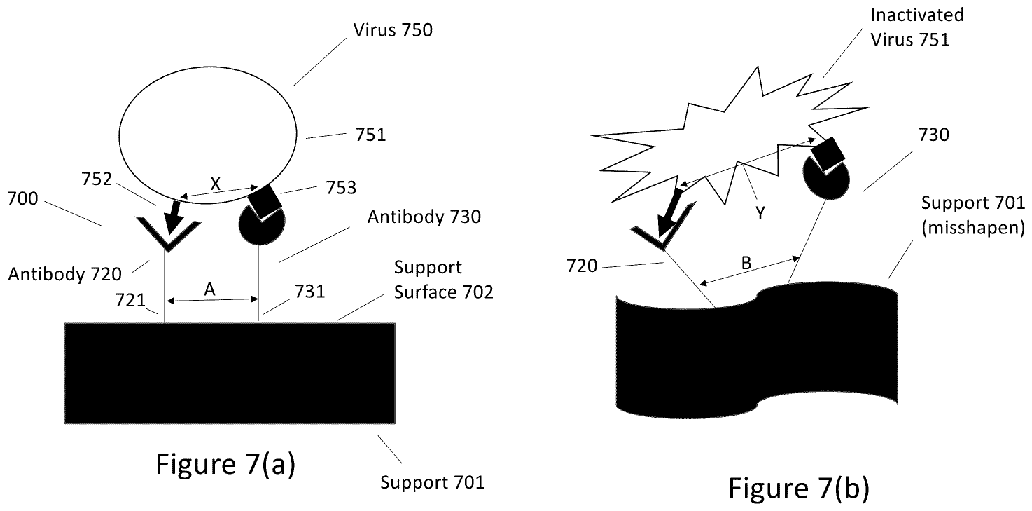

FIG. 7(a) shows another embodiment of the invention showing active complex 700 including a flat support 701 having support surface 702. Active complex 700 further comprises antibody 720 linked to support surface 702 at binding site 721 and antibody 730 linked to support surface 702 at binding site 731, preferably antibody 720 is adjacent antibody 730.

Also shown is virus 750 including virus membrane 751 and surface protein A 752 and surface protein B 753 on the surface of virus membrane 751. Antibody 720 is specifically bound to surface protein A 752 and antibody 730 specifically is bound to surface protein B 753.

Distance A is shown as arrows as the distance between antibody 720 and antibody 730 (e.g., distance between binding site 721 and binding site 731). Distance X is the distance between a point on the surface of virus membrane 751 (e.g., surface protein A 752) and another point on the surface of virus membrane 751 (surface protein B 753). In FIG. 7(a) the distances A and X are shown while support 701 is flat or not bent or not otherwise misshapen.

FIG. 7(b) shows active complex 700 and virus 750 of FIG. 7(a) after support 701 has been misshapen so no longer flat as shown. Distance B corresponds to distance A of FIG. 7(a) and is larger than distance A after the support 701 is misshapen, as shown. Similarly, distance Y is larger than distance X resulting in the tearing or otherwise destroying virus 750 as shown as inactivated virus 751. That is, as support 701 is misshapen, antibody 720 and antibody 730 are separated which, in turn, separates surface protein A 752 and surface protein B 753 sufficiently to tear, rupture or otherwise disrupt the virus membrane 751 of virus 750 forming inactivated virus 751.

Another embodiment of the invention relates to a therapeutic composition comprising at least a first antibody specific to the pathogen membrane (e.g., virus membrane) and a second antibody also specific to the pathogen membrane (e.g., specific to a different protein on the membrane surface) and the first antibody and second antibody are configured and/or orientated to disrupt, damage or destroy the membrane by movement or action of the first antibody relative to the second antibody, for example preferably by the flexing of the support, thereby destroying or inactivating the pathogen.

One embodiment of the invention relates to a therapeutic composition for treatment of at least one illness caused by a pathogen (e.g., virus) and/or symptoms, the composition comprising a particle having a first antibody or first antibody fragment that specifically binds to a first portion of pathogen (e.g., virus) and a second antibody or antibody second fragment that specifically binds to a second portion of the pathogen, preferably wherein the first portion is adjacent the second portion. Preferably, wherein the first antibody or first antibody fragment and the second antibody or antibody second fragment are configured to move the first portion of the pathogen relative to the second portion of the pathogen resulting in the pathogen membrane being disrupted or destroyed.

According to preferred embodiments, the first antibody and the second antibody are connected or attached or linked to a structure (e.g., particle, support, molecular structure), and the structure is configured to move the position or orientation or size of the first antibody relative to the second antibody to thereby disrupt, damage or destroy the pathogen membrane (e.g., virus membrane).

Another embodiment of the invention relates to a composition comprising at least one particle (e.g., microparticle, mesoparticle, nanoparticle, nanotube, carbon fibril, molecular sieve, etc.) and the therapeutic compositions described above. Preferably, the compositions described herein comprises a plurality of particles.

According to preferred embodiments, the particles used in the compositions described herein are nontoxic. Preferably, each particle is an environmentally compatible and/or biocompatible substance essentially nontoxic to human, animal and/or plant life.

Preferably, each particle is nontoxic to human, animal and/or plant life in the range of effective amounts used for treatments.

The particles used in the therapeutic compositions of the invention can have any suitable shapes and size depending on the formulation and method of administration and other factors. For example, the particle can have a shape of a sphere, square, rectangle, triangle, circular disc, cube-like shape, cube, rectangular parallelepiped (cuboid), cone, cylinder, prism, pyramid, right-angled circular cylinder and other regular or irregular shape.

As mentioned above, the shape, size, surface and composition of the particles used in any of the embodiments described herein should be optimized to increase the treatment effect while reducing any risks to the lower respiratory system or other areas of body.

For example, for eye drop formulations the particles are sized and shaped to avoid irritation to the eye, and preferably include additives specific for the application (e.g., lubricants for use in the eye, etc.). For example, the particles for use in eye drop formulations would have surfaces and edges that are preferably more smooth relative to particles administered orally or nasally.

The toxicity of a particle administered orally or nasally is largely dependent on particle size (Brook et al. Particulate matter air pollution and cardiovascular disease: An update to the scientific statement from the American Heart Association. Circulation. (2010); 121:2331-2378. doi: 10.1161/CIR.0b013e3181dbecel; Castranova V. Overview of current toxicological knowledge of engineered nanoparticles. J. Occup. Environ. Med. (2011); 53: S14-S17. doi: 10.1097/JOM.0b013e31821ble5a; Fubini B., Ghiazza M., Fenoglio I. Physico-chemical features of engineered nanoparticles relevant to their toxicity. Nanotoxicology. (2010); 4:347-363. doi: 10.3109/17435390.2010.509519), each hereby incorporated by reference in its entirety.

Carbon particles can be made as a pure carbon material, and thus has a relatively inert nature, which minimizes artifacts in toxicity caused by the chemical composition of particles.

The present invention further provides that in certain embodiments the particle has a diameter from about 10 μm to 100 mm. In certain embodiments, the diameter of the particle is about 50 μm to about 500 μm. In other embodiments, the diameter of the particle can be about 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 200 μm, 300 μm, 400 μm, 500 μm, 600 μm, 700 μm, 800 μm, 900 μm, 1 mm, 2 mm, 3 mm, 4 mm, 5 mm, 6 mm, 7 mm, 8 mm, 9 mm, and 10 mm, or any suitable sub-ranges within the about 10 μm to about 100 mm range.

According to preferred embodiments, the least one particle has a diameter (preferably an aerodynamic diameter) and/or largest dimension of greater than 10 μm and less than 100 mm, more preferably greater than 20 μm and less than 80 mm, even more preferably greater than 30 μm and less than 50 mm and most preferred greater than 100 μm and less than 10 mm.

According to preferred embodiments, the particle is not toxic to the human or animal in the effective amounts used during treatments. Preferably, the particles are biodegradable and biocompatible with humans and animals.

According one preferred embodiments, the particle is an oxide particle, preferably a silicon dioxide particle. Suitable particles also include silicon dioxide nanoparticles, such has those described in U.S. Pat. No. 10,111,952 to Weigandt et al.; WO 2012075087/U.S. Pat. No. 9,028,880 to Cheng at el.; WO2019016251 to Kraegeloh et al., each hereby incorporated by reference in its entirety.

According other preferred embodiments, the particle is water soluble, preferably a salt particle. According to alternative embodiments, the particle is a gelatin particle, a sugar particle or a flour particle. Preferably, the water-soluble particle comprises an outer coating that will slow the dissolving, melting, adsorption or other removal or elimination of the particle from the treatment area.

According to other preferred embodiments, the particle is a water-soluble polymeric particle.

One embodiment of the invention relates the use of carbon particles. Preferably, the particle is a carbon particle. According to other preferred embodiments, the particle is selected from the group consisting of graphene, carbon nanotubes (CNT)/carbon fibrils, buckyballs, buckytubes, carbon black and/or carbon quantum dots and/or combinations thereof.

According to other preferred embodiments, the particle has a plate-like shape (e.g., configured to adhere to surface of treatment area).

According to other preferred embodiments, the particle comprises activated carbon particles.

According to other preferred embodiments, the particle comprises carbon particles having metallic nanoparticles (e.g., gold) attached the surface, wherein the antibodies and/or surface-active agents are linked to the metallic nanoparticles.

According to other preferred embodiments, the particle has a tube-like elongated shape and/or cylindrical shape (e.g., configured to adhere to surface of treatment area and/or fit between cilia).

According to other preferred embodiments, the particle has a rectangular shape, preferably with one rounded side.

According to other preferred embodiments, the particle is an aggregation of nanoparticles (e.g., an assemblage of carbon nanotubes (“BN” or “bird nest” structure)) or an aggregation or cluster of nanoparticles. Complex structures of nanotubes have been obtained by linking functional groups on the tubes with one another by a range of linker chemistries. Known methods of functionalizing carbon particles are set forth in U.S. Pat. No. 7,578,989 to Moy, hereby incorporated by reference in its entirety. Agglomeration is a collection of primary particles, which involves diverse chemical and electrostatic forces such as van der Waals forces, magnetic forces and sintered bonds. Sonication is commonly and most frequently used in nanotechnology to disrupt such inter-particle interactions and thereby to disperse nanoparticles evenly in liquids.

Carbon fibrils have also been oxidized non-uniformly by treatment with nitric acid. International Application PCT/US94/10168 filed on Sep. 9, 1994 as WO95/07316 discloses the formation of oxidized fibrils containing a mixture of functional groups, hereby incorporated by reference in its entirety. Hoogenvaad, M. S., et al. (“Metal Catalysts supported on a Novel Carbon Support”, Presented at Sixth International Conference on Scientific Basis for the Preparation of Heterogeneous Catalysts, Brussels, Belgium, September 1994) describes oxidizing the carbon fibril surface with nitric acid, hereby incorporated by reference in its entirety.

According to preferred embodiments, the particles are three-dimensional assemblages of randomly oriented carbon fibril or carbon nanotubes, as described in U.S. Pat. No. 5,968,650 (JP2009167412) to Fisher et al., hereby incorporated by reference in its entirety, advantageously configured to provide increased surface area and/or provide increased adherence to area of treatment.

Functionalized nanotubes have been generally discussed in WO2006130150 to Hoch, U.S. Pat. No. 6,020,381 to Fisher, U.S. Pat. No. 6,031,711 to Tennent et al. and US20140162040 to Moy, each incorporated herein by reference in its entirety. In these applications the nanotube surfaces are first oxidized by reaction with strong oxidizing or other environmentally unfriendly chemical agents. The nanotube surfaces may be further modified by reaction with other functional groups. The nanotube surfaces have been modified with a spectrum of functional groups so that the nanotubes could be chemically reacted or physically bonded to chemical groups in a variety of substrates.

Carbon inks containing carbon particles have also been functionalized with antibodies and other biomaterials. For example, see the methods disclosed in U.S. Pat. No. 8,815,577 to Umek et al. teaching methods of immobilizing biomaterials on carbon ink electrodes using lectins or antibodies and also teaching methods of immobilizing antibodies on carbon, hereby incorporated by reference in its entirety. See also, U.S. Pat. No. 10,184,884 to Anderson et al., U.S. Pat. No. 8,808,627 to Wohlstadter et al. and U.S. Pat. No. 9,618,510 to Aghvanyan et al., each also describing methods of immobilizing antibodies on carbon and each hereby incorporated by reference in its entirety.

In yet certain other embodiments, the particle is a metallic particle, a polymeric particle, a dendrimer particle, or an inorganic particle.

In yet certain other embodiments, a gel or microgels are used (with or without particles). According to one preferred embodiment, the active agents (e.g., antibody, surface active agents) are suspended in a matrix (e.g., a gel) configured to allow the active agents to contact the virus particles either on the surface of the matrix and/or within the matrix (e.g., the virus can migrate through matrix to contact active agents) and/or the matrix dissolves or melts or disintegrates over time allowing the active agents to be released to contact and bind the virus particles. According to preferred embodiments, the therapeutic composition includes a gel manufactured from agarose or dextran or nitrocellulose or other cellulose.

In certain embodiments, the nanoparticle of the present invention is biocompatible and/or biodegradable.

Another aspect of the invention relates to therapeutic compositions comprising concave-shaped particles (e.g., bowl shaped) or particles having dimpled or porous surfaces. Specifically, according to particular embodiments, a component of the therapeutic composition may or will react or interact negatively with the human cells (e.g., a surfactant that might also react with cells, pathogen antibodies that might also bind with one or more cells and/or local bacteria), the component is advantageously embedded with a concave region on particle surface or within pore or within interior space(s) to prevent the component from contacting the human cell when the particle(s) are within the treatment area (e.g., upper respiratory system).

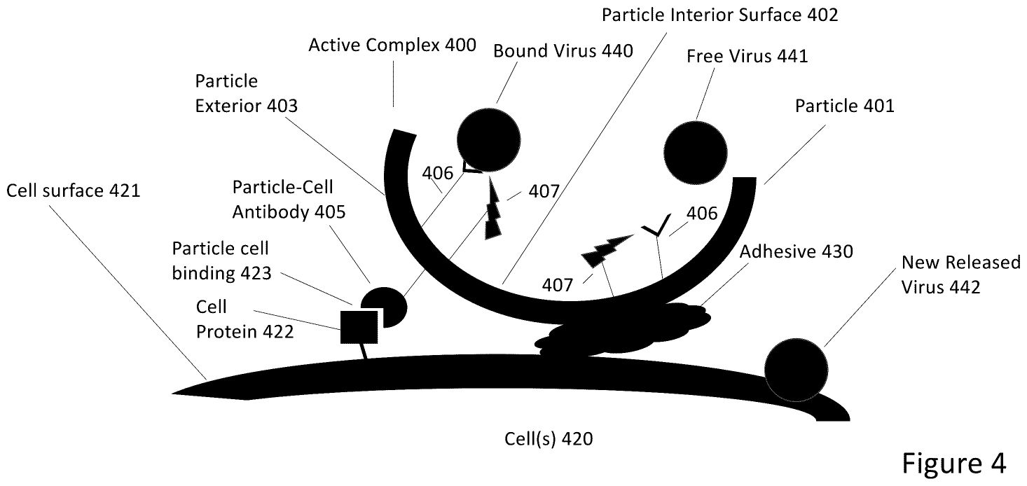

FIG. 4 is a graphical representation of another embodiment of the invention showing active complex 400 including a C-shaped particle 401 having a concave interior surface 402 and convex exterior surface 403. Active complex 400 further comprises antibody 406 which is specific to a target virus (e.g., free virus 441, released virus 442) and is immobilized on the concave interior surface 402 of particle 401. Active complex 400 further comprises surface-active agent 407 also immobilized on concave interior surface 402 and/or attached to antibody 406 (not shown).

Particle 401 is attached or immobilized or bound to the treatment area (shown as human cells 420) (and preferably for an effective amount of time for effective treatment and preferably only temporary) via adhesive 430 on the particle exterior surface 403 which adheres to cells 420 (or local bacteria, as described herein) and/or by antibody 405 on the particle exterior surface 403 that is configured to specifically bind to cell protein 422 at particle-cell binding site 423.

FIG. 4 also shows free virus 441 and also bound virus 440 with SAA 407 adjacent and close to inactivating the immobilized virus 440. FIG. 4 also shows newly released virus 442 from an infected cell (not shown). As shown, both bound virus 440 and free virus 441 are small enough to migrate into the concave side of particle 401 while cell(s) 420 are not able to do the same as a result of the C-shape (or bowl-shape) of particle 401. The C-shape of particle 401 allows the use of SAA molecules and/or antibodies on the interior surface 402 that might otherwise negatively affect the cell(s) 420. Particle 401 can alternatively be bowl-shaped, sphere-shaped, cube-shaped (with one or more openings to allow passage of virus particle), porous, voids, and an aggregate of particles (e.g., an assemblage of tangled nanofibers as shown in FIG. 5) or any other shape having one or more interior surface(s), void(s), pores, concave region(s), and/or one or more interior volumes that are accessible by pathogens (e.g., virus) but not accessible by human cell(s).

Preferably, the pathogen antibodies and/or any pathogen surface-active agents (e.g., surfactants) are attached within the concave surface and the second cell antibodies (e.g., anchor antibodies) and/or any adhesives are preferably on the outer convex surface. Preferably, the pathogen antibodies are on both surfaces, while pathogen surface-active agents only on the concave surface.

According to preferred embodiments, the least one particle has at least one concave surface and/or least one interior surface (e.g., pore) and the at least one amphipathic molecule and the at least one antibody or antibody fragment are connected to the least one concave surface and/or to the least one interior surface (e.g., pore).

According to another embodiment, the particle is a sphere with virus antibodies and anchor antibodies and/or adhesives around the entire surface, preferably spaced around the surface (vs covering each surface area). Preferably, the particle is configured to capture the pathogen (e.g., virus) with the antibody and sized/shaped to avoid the lower respiratory system and migrates into digestive system and/or is ejected by cough/sneeze. Preferably, the sphere-like particle has pores on its outer surface and the antibodies are immobilized within the pores.

According to another embodiment, the particle is plate-shaped with a mix of (i) pathogen antibodies and (ii) anchor antibodies and/or adhesives on both sides. Preferably, a plate-shaped particle advantageously sized and shaped to increase adherence to human cells, cilia and/or local bacteria.

According to particular embodiments, a component of the therapeutic composition may or will react or interact negatively with the cells (e.g., a surfactant that might also react with cells) and, according to such embodiment, the component is advantageously embedded within a concave region on particle surface or within pore or dimple within interior space(s).

Preferably, the at least one particle comprises a surface having a plurality of pores and the at least one amphipathic molecule and/or the at least one antibody or antibody fragment are connected to the interior of the pores (and/or dimples and/or concave region(s)).

Preferably, the pores, dimples and/or concave portions have diameters greater than 2 nm, preferably greater than 5 nm, more preferably greater than 10 nm since pores of 2 nanometers or less can be difficult to access because of diffusion limitations. Moreover, pores less than 2 nm are easily plugged and thereby deactivated. Therefore, pores mainly in the mesopore (>2 nanometers) or macropore (>50 nanometers) ranges are most desirable to avoid plugging and/or deactivation.

According to preferred embodiments, the particle comprises at least one interior volume having an interior surface and the at least one antibody structure is connected to the interior surface of the interior volume. Preferably, the particle comprises an outer surface and the outer surface includes at least one opening to the at least one interior volume. Preferably, the opening is greater than 2 nm, preferably greater than 5 nm, more preferably greater than 10 nm. Preferably, the opening is sized to be larger than the average largest diameter of the pathogen of interest to allow the pathogen to enter through the opening.

Preferably, the particle is a porous carbon fibril/nanotube structure having a high surface area (preferably greater than about 100 m²/gm) substantially free of micropores, for example, the structures described in U.S. Pat. No. 6,960,389 to Tennent et al, hereby incorporated by reference in its entirety. High surface area particles would advantageously allow for the use of more antibodies and/or surface-active agents on the surface. According to preferred embodiments, each of the at least one particle has a second surface opposing the outer surface and wherein the second surface is configured to be mucosa-adhesive and/or comprises mucosa-adhesives.

FIG. 5 is a graphical representation of another embodiment of the invention showing active complex 500 immobilized on cell surface 521 of cell(s) 520 (e.g., cells of treatment area and/or local bacteria). Active complex 500 comprises a support 501 composed of an assemblage of entangled nanofibers 502 resulting in support voids 550 distributed throughout the support 501. Active complex 500 further comprises antibody 507 configured to specifically bind virus 1. Active 500 further comprises surface-active agents 511 configured to destroy, disrupt or otherwise inactive virus 1. Surface-active agents 511 can be immobilized on support 501 adjacent to antibody 507 and/or on separate portions of support 501 (as shown).

Active complex 500 also comprises antibody 560 configured to specifically bind virus 1 to support 501 to immobilize virus 1 (and subsequently expel from treatment area via digestive system or flushing or coughing or sneezing).

Active complex 500 further comprises antibody pairs 550 configured to bind to two different locations on the same virus membrane and then disrupt or destroy or otherwise inactivate the virus when the nanofiber 502 flexes or bends separating and/or changing the orientation of antibody pair 550 and at same time separating the two different locations of the virus member and thus inactivating the virus.

The active complex 500 shown in FIG. 5 includes two mechanisms to immobilize or adhere the active complex 500 to cell surface 511. According to invention, active complexes can include both mechanisms and/or each mechanism alone and/or additional mechanism (e.g., mechanical features to interlock with cilia or other morphologies within treatment area). The active complex 500 includes particle-cell antibody 510 configured to specifically bind to binding protein 523 on cell surface 521 and/or also comprises adhesive 530 to adhere support 501 to cell surface 521 for an effective time for treatment.

Preferably, support 501 comprises a plurality of antibodies 507 and SAA 511 and/or antibody pairs 550 distributed throughout the support 501.

Preferably, adhesive 530 is distributed throughout support 501 to increase likelihood adhesive contacts to a surface within the treatment area (e.g., cell surface 521).

Preferably support 501 comprises a plurality of particle-cell antibodies 510 to increase likelihood specifically binds to a surface within the treatment area (e.g., cell surface 521). According to preferred embodiments, different particle-cell antibodies 510 are used (e.g., specific for different binding sites on cell surface 521).

Preferably, support 501 comprises support voids 550 throughout as shown to allow virus 1 to migrate within the support 501 and contact the one or more active agents within the support 501 (e.g., antibody pair 550, antibody 560 and/or antibody 507-SAA 511).

Advantageously, support 501 as shown in FIG. 5 would provide increased surface area for immobilizing a higher number of active agents compared to a solid particle. The overall structure of support 501 preferably includes a sheet or flat structure composed of the nanofibers or a particulate shape (with the preferred particle dimensions described herein).

According to particularly preferred embodiments, the particles used in the compositions described herein have a high surface area, preferably greater than about 10 m²/gm, more preferably greater than about 50 m²/gm, even more preferably greater than about 100 m²/gm and most preferred greater than about 200 m²/gm. Having a high surface area allows a greater number of pathogen antibodies, anchor antibodies and/or surface-active agents to be associated with each particle (e.g., a higher load of active agents (e.g., antibodies, surfactants, etc.)).

Preferably, at least 50% of the surface area of the particles comprises pathogen antibodies, anchor antibodies and/or surface-active agents (e.g., surfactant), more preferably at least 75%, even more preferably at least 90% and most preferred at least 99%.

According to other embodiments of the invention, the particles are optimized and/or configured to connect/attach or within between the cilia in the treatment area. Cilia are elongated hair-like structures on surfaces within the upper respiratory system to facilitate the removal of particulate matter. The length of a single cilium is 1-10 microns and the width is less than 1 micron. ‘Motile’ (or moving) cilia are found in the lungs, respiratory tract and middle ear. These cilia have a rhythmic waving or beating motion.

Preferably, the particles comprise ligands capable of adhering or attaching to cilia and/or are configured to clasp onto the cilia. According to preferred embodiments, each particle comprises branches or ‘tentacles’ (e.g., a nanotube aggregate particle having loose nanotube ends emitting therefrom, a particle having a ‘star-like’ or octopus-like shape, particles having supramolecules attached thereto) to at least temporarily immobilize the particle onto the cilia and/or group of cilia either chemically (e.g., via chemical bonds) and/or mechanically (e.g., loose nanotube end engaging or wrapping around cilia). According to preferred embodiments, the branches or “tentacles” comprising adhesive on the surface and/or distal ends to facilitate adhesion.

According to particularly preferred embodiments, the particles are configured to fit between cilia to attach or bind to cell surface of treatment area.

According to preferred embodiments of the invention, the therapeutic compositions described herein include at least one antibody or fragment thereof specific to the pathogen of interest (or allergen or other foreign substance).

Preferably, the antibody (or “binding effector molecule”) is a target-binding peptide or polypeptide is specific for a pathogen, preferably a pathogen having an outer membrane (e.g., an enveloped virus).

Preferably, the antibody (or “binding effector molecule”) is specific for the pathogen membrane, more preferably for a protein of a virus having a lipid bilayer.

The terms “antibody or a fragment thereof” refer to molecules having immunoreactivity to an analyte of interest, for example, a pathogen (e.g., virus). In further embodiments, the antibody is a monoclonal antibody, a chimeric antibody, a humanized antibody, a human antibody, a bispecific antibody, or an antibody fragment. In some embodiments, the therapeutic polypeptide is produced in mammalian cells. In some embodiments, the mammalian cell line is a Chinese Hamster Ovary (CHO) cells, or baby hamster kidney (BHK) cells, murine hybridoma cells, or murine myeloma cells.

The term “monoclonal antibody” as used herein preferably refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (e.g., epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present invention may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567), each hereby incorporated by reference in its entirety. The “monoclonal antibodies” may also be isolated from phage antibody libraries using the techniques described in Clackson et al., Nature, 352:624-628 (1991) and Marks et al., J. Mol. Biol., 222:581-597 (1991), for example, each hereby incorporated by reference in its entirety.

The monoclonal antibodies herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81:6851-6855 (1984)), each hereby incorporated by reference in its entirety. Chimeric antibodies of interest herein include “primatized” antibodies comprising variable domain antigen-binding sequences derived from a non-human primate (e.g., Old World monkey, ape etc.), and human constant region sequences.

“Humanized” antibodies are forms of non-human (e.g., rodent) antibodies that are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992), each hereby incorporated by reference in its entirety.

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.); PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty, ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993), each hereby incorporated by reference in its entirety.

According to certain embodiments of the invention, the composition includes at least an additional antibody specific to one or more cells used to adhere the particle to the interior surface of the throat or nasal passages (e.g., anchor antibody) to allow the components on the particle to perform the treatment.

According to additional embodiments, the composition includes at least one additional antibody which is specific to one or more local bacteria (e.g., an anchor antibody specific to local bacteria). According to additional embodiments, the composition further includes at least one additional antibody which is specific to one or more local bacteria, in addition to antibody specific to one or more cells to result in at least two different antibodies to act as anchor antibodies. Preferably, the composition includes one or more bacterial-specific ligands for the one or more local bacteria, preferably one or more of such bacterial-specific ligands connected to the one or more particles.

According to alternative embodiments, the anchor antibody is specific to the local bacteria instead of the cells. Mucosal membranes are colonized with large numbers of resident commensal bacteria (herein “local bacteria”). If the particles are immobilized onto the surface of these mucosal bacteria, the “half-life” of the particles within the area of treatment will be significantly improved. First, particles immobilized onto mucosal bacteria would be much less likely to be flushed out by the mucociliary clearance mechanisms on the mucosa, as described in U.S. Pat. No. 6,365,156 to Lee, hereby incorporated by reference in its entirety. Since bacteria are generally considerably larger than viral particles, the viral particles will be prevented from moving on to infect host cells since “tied to” the larger bacteria or at least reduced. A variety of different bacteria can be targeted. For example, the viral-specific ligand can be modified to bind to Streptococcus, Lactobacillus, Streptococcus, Staphylococcus, Lactococcus, Bacteroides, Bacillus, and Neisseria. The particle can be modified using a bacterial-specific ligand that is an antibody, a polypeptide, a protein, a peptide, a lipid, or a carbohydrate, or combination thereof, specific for a component of the extracellular material of the bacteria. Alternatively, one can use antibody fragments, single chain antibodies, F(ab)s, F(ab)2s or bacterial specific non-antibody binding elements. The bacterial-specific ligand can also be selected from the group consisting of: a C-terminal choline binding domain of LytA, a C-terminal binding domain of PspA, a C-terminal domain of lysostaphin (SPAcwr), a C-terminal domain of InIB, an anti-S-layer protein antibody, and an anti-peptidoglycan antibody. The viral-specific ligand and the bacterial-specific ligand can be joined by a variety of means. These include bifunctional linkers both hetero and homobifunctional linkers and peptide linkers.

Another alternative embodiment of the invention employs an anchor antibody specific to the local bacteria and the anchor antibody specific to the local bacteria is linked, either directly or indirectly, to at least one antibody specific for the pathogen and/or one or more surface active agents, as described here.

According to a further alternative embodiment of the invention, at least one local bacteria is modified to include one or more pathogen-specific and/or foreign substance-specific antibodies and/or any pathogen surface-active agents (e.g., surfactants), preferably immobilized on the surface of the local bacteria. Preferably, the antibodies and/or surface-active agents are configured with one or more linkages to attach to the local bacteria. That is, the local bacteria is used as the “support” for the antibodies and/or surface-active agents. According to one embodiment, the antibodies and/or surface-active agents are administered and immobilized on the local bacteria within the treatment area (e.g., upper respiratory system). According to another embodiment, the antibodies and/or surface-active agents are immobilized on the local bacteria and the modified local bacteria are administered to the treatment area.

Another aspect of the invention relates to destroying, disrupting or inactivating a pathogen membrane (e.g., virus membrane) using antibodies and surface-active agents (including surfactants or amphiphile molecules) and methods of using the same. Preferably, therapeutic compounds, compositions and/or structures that include one or more molecules configured to destroy the outer membrane shell of pathogen (e.g., virus or substance) (e.g., “surface-active agents”).

It is known that washing hands well with soap works well in destroying the Covid-19 virus and other viruses, and other pathogens. Typically, the soap includes surfactant molecules that disrupt the virus membrane. Surfactant molecules used in soap typically include a hydrophilic head and a hydrophobic tail (i.e., the head likes to bond to water while the tail is hydrophobic so rejects water). The hydrophobic tail is drawn to the fatty outer layer of the virus and which will pry the virus shell open. Once the virus (or bacteria) splits open, it spills its RNA or DNA guts and dies.

One aspect of the invention relates to surface-active agents including surfactants, molecules having an amphipathic structure and other molecules designed and configured to be capable of disrupting the pathogen (e.g., virus membrane), that is the invention is not limited to amphiphile molecules but any molecule capable of destroying or disrupting the membrane of a virus.

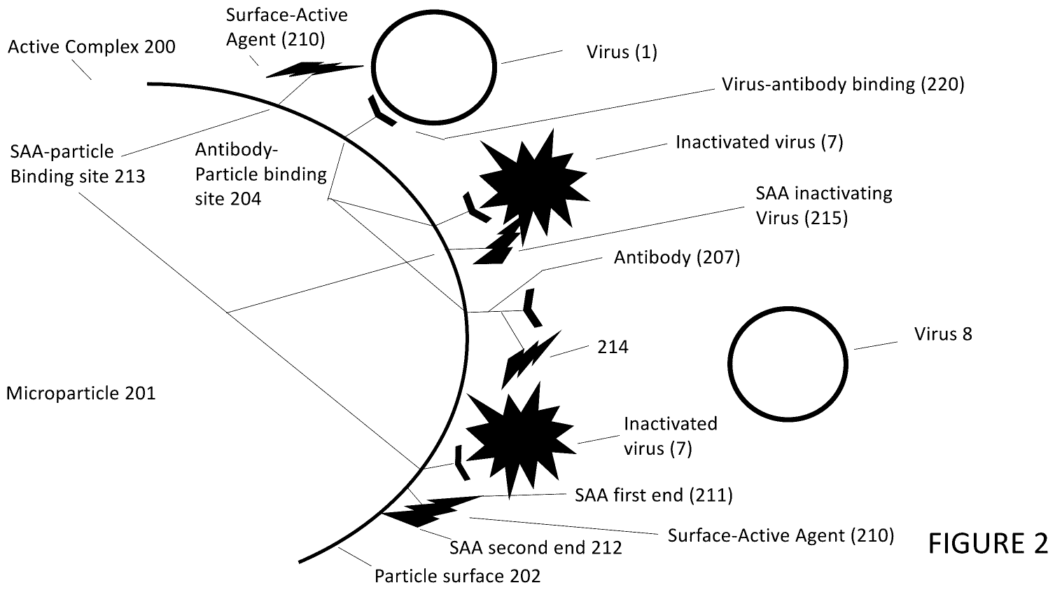

FIG. 2 is a graphical representation of one embodiment of the invention showing active complex 200 comprising microparticle 201 having particle surface 202. Active complex 200 also includes antibodies 207 immobilized on particle surface 202 at antibody-particle binding sites 204 and surface-active agents 210 (“SAA”) immobilized at SAA-particle binding sites 213 and surface-active agent 214 immobilized on antibody 207 (or immobilized on a linkage attaching antibody 207 to antibody-particle binding site 204).

FIG. 2 also shows virus 1 immobilized on an antibody 207 and free virus 8 not yet immobilized by active complex 200.

Surface-active agents or SAA 210/214/215 are configured to destroy, disrupt, rupture or otherwise inactivate the viruses (e.g., virus 1), preferably one or more amphiphile molecules configured to target the virus or the virus membrane. SAA 210/214 preferably comprises SAA first end 211 and SAA second end 212. SAA second end 212 is preferably hydrophilic and preferably attracted to particle surface 202 and/or liquid environment at the treatment area (not shown). SAA first 211 is preferably hydrophobic and configured and/or selected to pierce the virus membrane. FIG. 2 also shows inactivated virus 7 after inactivation by a SAA 215.

According to one preferred embodiment, the invention employs or uses a soap-like molecule or amphiphile molecule selected and/or configured to preferentially destroy the membrane shell of a virus, specifically, a “soap-like” molecule that targets and, preferably only cleaves open the targeted virus membrane to destroy it (and preferably does not interact with human cells). For example, a molecule having a first end which is hydrophilic and a second end which is hydrophobic.

Preferably, one end of the molecule is configured to be attracted to the environment (e.g., liquid, mucous) within the nasal passages, throat or the extracellular fluid within the bloodstream, while the other end is configured to attracted to the pathogen membrane (e.g., virus membrane, protein on virus surface) and/or otherwise configured to destroy the pathogen membrane or pathogen.

Another aspect of the invention relates to compositions comprising the surface-active agent (e.g., surfactant) and one or more antibodies having specificity to the pathogen of interest.

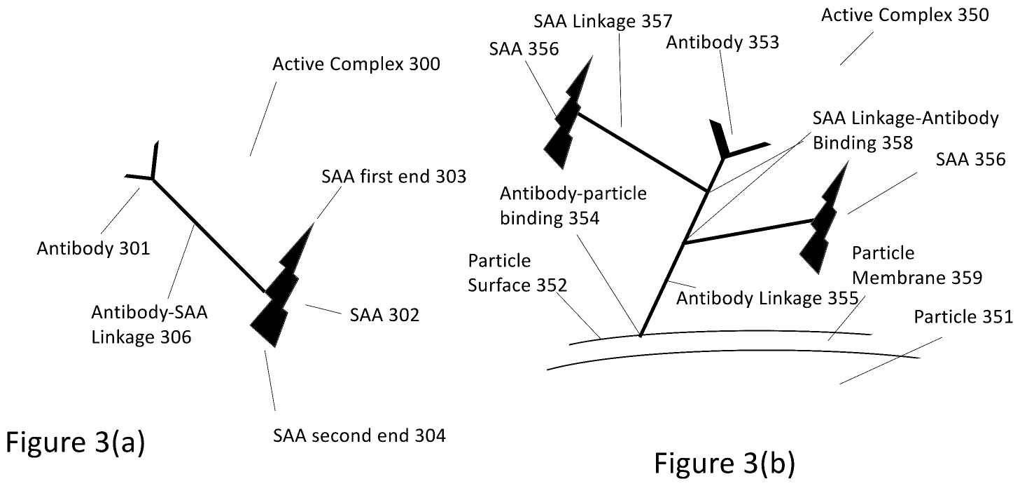

FIG. 3(a) is a graphical representation of another embodiment of the invention showing active complex 300 comprising a surface-active agent 302 linked to antibody 301 via linkage 306. Surface-active agent (or “SAA”) 302 includes SAA first end 303 and SAA second end 304. SAA first end 303 is preferably hydrophobic or otherwise configured/active to disrupt or destroy or otherwise inactivate a pathogen, preferably the membrane of a pathogen (not shown). Active complex 300 does not require a particle. According to preferred embodiments, active complex 300 is embedded or immobilized on and/or within a film, gel, paste, foam, or other formulation applied to a treatment area or embedded or immobilized on a component inserted or applied to a treatment area or infected area, for example, coated on the outside or immobilized on the outer surface of an endotracheal tube (ET tube), discussed further below. According to an alternative embodiments, active complex 300 is administered to the lower respiratory system or other treatment area since particles are not used, particularly SAA 302 is preferably an acceptable natural surfactant is employed is the active complex 300 for treatment of areas outside upper respiratory system (e.g., upper and/or lower respiratory system).

FIG. 3(b) is a graphical representation of another embodiment of the invention showing active complex 350 comprising particle 351 and antibody 353 immobilized on particle surface 352 at binding site 354. FIG. 3(b) shows antibody linkage 355 linking antibody 353 to particle 351 (alternatively, antibody 353 can be immobilized on particle surface 353 without linkage 355). FIG. 3(b) shows two SAA 356 molecules attached to antibody 353 via SAA linkage 357 at SAA linkage-antibody binding sites 358. As shown, each SAA 356 is preferably configured adjacent to the antibody binding sites where the virus (not shown) will be bound facilitating the inactivating of the virus by either or both SAA 356 molecules.

According to alternative embodiments, antibody 353 or Active Complex 300 can be linked or attached or connected to a cell (e.g., ciliated cells in the superficial epithelium and/or type-2 pneumocyte cells) and/or local bacteria instead of being attached to particle 351 allowing the particle to be omitted from the therapeutic composition.

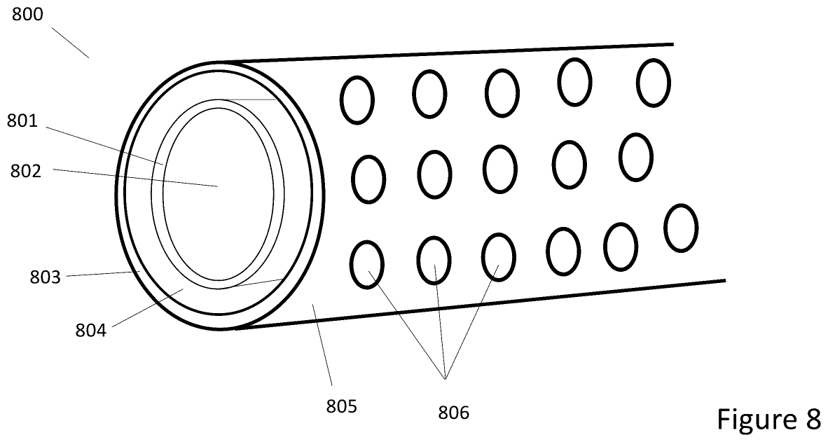

According to further alternative embodiments, antibody 353 or Active Complex 300 or Active Complex 350 can be linked or attached or connected or embedded or absorbed or adsorbed into and/or onto a band aid, bandage or other wound dressing or device configured to insert into a body (e.g., surgical instrument or ET tube shown in FIG. 8 or the like) (described below).

Preferably, the surface-active molecule (e.g., surfactant) is attached or linked to an antibody that is configured to specifically bind the pathogen (e.g., virus membrane).

According to further alternative embodiments, the therapeutic compositions comprise a mixture of the therapeutic compositions described herein. Preferably, a first therapeutic composition portion comprising antibody-particle complex and a second therapeutic composition portion comprising the antibody-amphiphile molecule(s) complex to provide synergies during treatment.

One embodiment of the invention relates to a therapeutic composition for treatment of an illness caused by a virus or other pathogen or foreign substance, the composition comprising at least one amphiphile molecule and at least one antibody structure comprising an antibody or antibody fragment that specifically binds to the pathogen (e.g., virus) or substance.

Preferably, wherein the at least one amphiphile molecule is connected to the at least one antibody structure.

Preferably, the at least one amphiphile molecule is connected adjacent to at least one binding site of the at least one antibody structure.

Preferably, the at least one amphiphile molecule is connected to the Fab region of the at least one antibody structure.

Preferably, the at least one amphiphile molecule is connected to the Fc region of the at least one antibody structure.

Preferably, the therapeutic composition further comprises at least one second antibody structure comprising a second antibody or a second antibody fragment specific to one or more cells that line the nasal passages, oral cavity and/or throat and/or specific to one or more local bacteria that line the nasal passages, oral cavity and/or throat.

According to preferred embodiments, the composition further comprises at least one second antibody structure comprising a second antibody or a second antibody fragment specific to epithelium cells or squamous cells.

Preferably, the at least one second antibody structure is configured to attach the at least one amphiphile molecule and the at least one antibody structure to one or more sides of nasal passages, oral cavity and/or throat.

According to preferred embodiments, the therapeutic compositions described herein are environmentally compatible and/or biocompatible compositions/substances essentially nontoxic to human, animal and/or plant life.

Preferably, the therapeutic compositions described herein are nontoxic to human, animal and/or plant life in the effective amounts needed for the treatment application.

Preferred embodiments of the invention include at least one amphiphile (e.g., surfactant) capable of interacting and otherwise destroying the membrane of the virus or other pathogen.

According to preferred embodiments, the at least one amphiphile molecule comprises at least one hydrophilic end and at least one hydrophobic end.

According to preferred embodiments, the at least one amphiphile molecule comprises a hydrophilic end and a hydrophobic end.

According to preferred embodiments, the at least one amphiphile molecule comprises at least one hydrophobic end, and, according to alternative embodiments, does not include a hydrophilic end but instead optionally attached or connected or adsorbed onto a micro particle or other structure.

According to one embodiment, the at least one amphiphile molecule comprises a hydrophilic end and a hydrophobic end and the at least one antibody structure is connected to the at least one amphiphile molecule between the hydrophilic end and the hydrophobic end.

According to another embodiment, the at least one amphiphile molecule comprises a hydrophilic end and a hydrophobic end and the at least one antibody structure is connected to the hydrophilic end of the at least one amphiphile molecule.

According to another embodiment, the at least one amphiphile molecule comprises a hydrophilic end and a hydrophobic end and the at least one antibody structure is connected to the hydrophobic end of the at least one amphiphile molecule.

In some preferred embodiments of the invention, the surfactant is a non-ionic surfactant or zwitterionic surfactant. In some additionally preferred embodiments, the surfactant has a hydrophile lipophile balance (HLB) from about 12 to about 15.

In some preferred embodiments of the invention, the surfactant does not comprise or form octylphenol. In some preferred embodiments, the surfactant does not comprise or form peroxide.

In some preferred embodiments of the invention, upon disposal the surfactant's Predicted Environmental Concentration (PEC) in a receiving water body resulting from use of the surfactant is below the Predicted No-Effect Concentration (PNEC) of the surfactant. In further preferred embodiments, the PEC is lower than the PNEC.

In some preferred embodiments of the invention, the environmentally compatible surfactant comprises an alkyl glucoside. In further embodiments, the alkyl glucoside is a decyl glucoside. In other preferred embodiments, the environmentally compatible surfactant is an alcohol ethoxylate. In yet other preferred embodiments, the environmentally compatible surfactant is an alkyl polyethylene glycol ether. In some preferred embodiments, the environmentally compatible surfactant has a CAS registry number of CAS 9005-64-5, CAS 9005-65-6. CAS 126-43-8, 68515-73-1, CAS 58846-77-8, CAS 59122-55-3, CAS 110615-47-9, CAS 29836-26-8, CAS 64366-70-7, CAS 68937-66-6, CAS 69227-22-1, CAS 25322-68-3, CAS 27252-75-1, CAS 4292-10-8, CAS 132778-08-6, CAS 110615-47-9, CAS 68515-73-1, or CAS 68439-46-3.

According to particularly preferred embodiments, the at least one amphiphile molecule comprises at least one natural surfactant found in individuals, more preferably at least one lung surfactant and/or pulmonary surfactant (e.g., dipalmitoylphosphatidylcholine (DPPC) CAS No. 2644-64-6)), even more preferably at least one natural surfactant found in alveoli. Pulmonary surfactant is a surface-active lipoprotein complex formed by type II alveolar cells. The proteins and lipids that make up the surfactant have both hydrophilic and hydrophobic regions. One advantage of using a natural surfactant is reducing risks of toxicity, bioactivity, or related risks. According to another embodiment, a synthetic version and/or modified version of the natural surfactant is used in the therapeutic composition.

Preferably, the length and configuration/structure of the amphiphile molecule is configured to contact the outer membrane of the pathogen membrane (and preferably pry the membrane open or otherwise destroy the membrane or pathogen) and, more preferably not capable of interacting with cell.

Preferably, the composition further includes at least particle, such as the particles described herein. According to preferred embodiments, the amphiphile molecule is configured to only reach up to 10 mm from the surface of the particle where the molecule is attached, preferably less than 5 mm, even more preferably less than 2 mm, even more preferably less than 1 mm, and even more preferably less than even more preferably less than 0.1 mm.

According to further preferred embodiments, the amphiphile molecule is configured to only reach up to 10 microns from the surface of the particle where the molecule is attached, preferably less than 5 microns, even more preferably less than 2 microns, even more preferably less than 1 microns, and even more preferably less than even more preferably less than 0.1 microns.

For example, coronaviruses are pleomorphic spherical particles with bulbous surface projections. The average diameter of the virus particle is around 120 nm (0.12 μm). The diameter of the envelope is ˜80 nm (0.08 μm) and the spikes are ˜20 nm (0.02 μm) long. The viral envelope consists of a lipid bilayer where the membrane (M), envelope (E) and spike (S) structural proteins are anchored. According to particularly preferred embodiments, the amphiphile molecule is configured to only reach up to 10 microns from the surface of the particle where the molecule is attached, preferably less than 5 microns, even more preferably less than 2 microns, even more preferably less than 1 micron, and even more preferably less than even more preferably less than 0.1 micron. Preferably, the amphiphile molecule is designed and configured to contact the membrane envelope of the pathogen while the pathogen is attached to the particle via the antibody, preferably while avoiding contact with cells of the host.

Preferably, the composition comprises one or more antibodies having a length ranging from 5-15 nm (preferably about 10 nm) attached to the particle and the one or more the amphiphile molecules also attached to the particle is configured reach 5-15 nm (preferably about 10 nm).

According one embodiment, the amphiphile molecule is attached or connected to the particle by a tethering molecule whereby one end of the tethering molecule is attached to the particle and/or the pathogen antibody structure and the other end of the tethering molecule is attached or connected to one or more amphiphile molecules. Preferably, the tethered amphiphile molecule is designed and configured to contact the membrane envelope of the pathogen while the pathogen is attached to the particle directly or via the antibody.

According another embodiment, the amphiphile molecule is attached or connected to the pathogen antibody by a tethering molecule whereby one end of the tethering molecule is attached to the pathogen antibody structure and the other end of the tethering molecule is attached or connected to one or more amphiphile molecules. Preferably, the tethered amphiphile molecule is designed and configured to contact the membrane envelope of the pathogen while the pathogen is attached to the antibody, preferably while avoiding contact with cells of the host.

According one embodiment, the amphiphile molecule is attached or connected to the particle by a tethering molecule whereby one end of the tethering molecule is attached to the particle and the other end of the tethering molecule is attached or connected to one or more amphiphile molecules. Preferably, the tethered amphiphile molecule is designed and configured to contact the membrane envelope of the pathogen while the pathogen is attached to the particle.

Preferably, the tethering molecule is at least one ligand and/or at least one bifunctional linking reagent and/or at least one bifunctional linker. One of ordinary skill in the art could select and configure the tethering molecule in view of the description set forth herein.

Preferably, the tethering molecule has a length between 1 nm and 1000 nm, more preferably between 5 and 500 nm, even more preferably between 10 and 100 nm, and even more preferably between 15 and 50 nm.

Preferably, the length of the tethering is optimized and/or configured to optimize the range of the “working” end of the surface-active molecule (e.g., surfactant) considering whether the surface-active molecule is attached directly to the particle or pathogen antibody or tethered to one or the other and depending on the surface morphology of the treatment area (e.g., presence of cilia).

According to one embodiment, the particle is a nanocage having an interior volume and an exterior surface, wherein the at least one amphiphile molecule and the at least one antibody or antibody fragment are attached within the interior volume(s) of the nanocage. Preferably, the at least one amphiphile molecule and/or the at least one antibody or antibody fragment are attached to an inner surface within the interior volume of the nanocage and, preferably, the pathogen can access the interior volume of the nanocage.

Preferably, the nanocage comprises one or more openings to the interior volume.

Preferably the nanocage comprises an outer surface having at least one second antibody or second antibody fragment specific to one or more cells that line the nasal passages, oral cavity and/or throat (for example, epithelium cells, squamous cells).

According to one embodiment, each of said at least one particle has an outer surface and wherein the at least one amphiphile molecule and the at least one antibody structure are attached, directly and/or indirectly, to the outer surface. Preferably, each of the at least one particle further comprises at least one second antibody structure comprising a second antibody or a second antibody fragment specific to one or more cells (and/or local bacteria) that line the nasal passages, oral cavity and/or throat (for example, epithelium cells, squamous cells).

According to preferred embodiments, the outer surface of the particle repels the hydrophobic end of the at least one amphiphile molecule, preferably the outer surface is hydrophilic or superhydrophilic.

Preferably, the at least one particle comprises an outer surface, and the outer surface has been oxidized (e.g., oxidized carbon nanotubes or oxidized carbon fibrils).

Preferably, each of said at least one particle comprises an outer surface, and the outer surface has been oxidized and functionalized by chemical substitution or by adsorption of functional moieties (e.g., functionalized nanotubes as described herein).

Preferably, molecule is configured so that the hydrophobic end or “working end” is not adsorbed onto or absorbed into the particle; instead it should be configured to be “floating” or otherwise available to contact the virus particle during or after the virus particle binds to the antibody, for example.

According to preferred embodiments, the hydrophobic end of the amphiphile molecule is more strongly hydrophobic than the hydrophilic end is hydrophilic. Thus, the hydrophilic end controls the position of the amphiphile molecule without the presence of something to attract the hydrophobic end (e.g., without the presence of the virus membrane). Preferably, the surface of the particle is hydrophilic and thus attracts the hydrophilic end of the amphiphile molecule. However, when the antibody binds the pathogen (e.g., virus) proximate to the amphiphile molecule, the hydrophobic end of the amphiphile molecule is drawn to the fatty outer layer of the pathogen and pries the membrane shell open or otherwise inactivates the virus. Once the pathogen (e.g., virus) splits open, it spills its RNA or DNA guts and dies. That is, the binding of the pathogen to the particle “activates” the amphiphile molecule to orient and/or move the hydrophilic end towards the membrane of the pathogen to disrupt, destroy and/or otherwise inactivate the membrane.

As mentioned above, preferably the soap-like molecule is attached to a structure (e.g., the inner surface of C-shaped structure or within a cage-like particle) that preferably cannot reach or touch the cell (i.e., cell too large for structure) but the virus can fit within the structure and/or concave surface and/or interior void(s), allows the soap molecule to contact and split open the membrane of the pathogen and/or virus. That is, the structure prevents the soap-molecule or other surface-active agent from contacting the cell membrane but allows contacting the pathogen and more preferably attracts the virus.

For example, a molecular sieve with the “soap-like molecule” attached within the sieve and with openings allowing the virus to enter and contact soap-like molecule. Preferably the sieve has antibodies specific to virus to attract and hold virus preferably within the interior openings. According to another embodiment, a nanotube (e.g., carbon nanotube), nanocage (e.g., “gold nanocage”), nanoassembly or other nanostructure is used as the platform for the soap-like molecule. Preferably, the nanostructure further comprises antibodies that are configured to be specific for the pathogen (e.g., surface of the virus).

For example, a nanoparticle having a surface wherein the surface has one or more antibodies specific to the virus attached thereto and one or more “soap-like molecules” also attached to the surface to pierce virus membrane. The antibodies are adapted to attach the virus to the nanoparticle. And the soap-like molecule configured to (including length or reach of molecule) to pierce virus membrane. Preferably, the nanoparticle comprises a plurality of antibodies and a plurality of soap-like molecules, each dispersed around the nanoparticle surface.

According to one preferable embodiment, the nanoparticle has a first side and an opposing second side, wherein the first side surface is populated with pathogen antibodies and surface-active agents (e.g., soap-like molecules) and the opposing second side is configured to attach to side of throat and/or nasal passages (e.g., mucosa-adhesives, anchor antibodies).

According to another embodiment, the nanoparticle surface containing the antibodies and soap-like molecules is pitted or porous and the antibodies and the surface-active agents (e.g., soap-like molecules) are attached within the pits/pores or other void(s), wherein the pits/pores are large enough to allow the pathogen (e.g., virus) to enter and contact the antibodies and/or agents.

According to another embodiment, the nanoparticle surface containing the antibodies and soap-like molecules is concave allowing the virus to contact the surface but too narrow to allow a human cell(s) to contact.

Another embodiment of the invention relates to the use of particles (e.g., salt nanoparticle) in the compositions that dissolve (e.g., dissolve in the upper respiratory system) over a period of time, preferably dissolves between 1-3 hours, more preferably 3-6 hours and more preferably 6-12 hours. Preferably, the particle dissolves in the nasal passages, oral cavity and/or throat over a period of time. Preferably the particle dissolves in the throat over a period of time (e.g., salt particle).

Another embodiment of the invention relates to the use of particles that will disintegrate or melt (e.g., disintegrate/melt in the upper respiratory system) over an effective amount of time (e.g., ice microparticles, salt particles, edible particles) including the times periods recited above regarding particles that dissolve.

According to an alternative embodiment, a gel or gel particles are used as “particles” and the active agents are suspended in the gel or gel matrix.

Another embodiment of the invention relates to therapeutic formulations (e.g., liquids, syrups, powders, aerosols) comprising a therapeutic composition as described herein and one or more liquids and/or one or more additives adapted for use in therapeutic applications.

Preferably, the therapeutic composition is dispersed in a liquid, wherein liquid formulation is configured for administering to a subject topically, by injection, to the eye, orally, or by inhalation.

According to preferred embodiments, the formulation is a syrup. That is, the formulation has an approximate viscosity (at room temperature (70° F.)) ranging from about 10 centipoise (or cps) to about 100,000 cps, preferably from about 500 cps to about 50,000 cps and most preferably about 1,000 cps to about 25,000 cps (e.g., measuring using a conventional viscometer (e.g., EMS-1000)).

According to preferred embodiments, the formulation is thixotropic. That is, gels or foams or fluids that are thick or viscous under static conditions that will flow (become thinner, less viscous) over time when shaken, agitated, shear-stressed, or otherwise stressed (time dependent viscosity). For example, a formulation that has lower viscosity during application (e.g., while being sprayed through a nozzle) but has higher viscosity after applied to an upper respiratory surface.

According to preferred embodiments, the formulation comprises (i) water; (ii) at least one film forming agent; (iii) at least one emulsifier and/or thickening agent; and (iv) at least one antimicrobial agent.

Preferably, the formulation includes one or more of the following additives: water; sweetener (e.g. propylene glycol, sucralose, glycerin); emulsifier or thickening agent (e.g., xantham gum); pH adjustment agent (e.g., anhydrous citric acid); preservative (e.g., methylparaben); disintegrant (e.g., carboxymethylcellulose sodium or sodium glycolate); antimicrobial (e.g., sodium benzoate); binding agent (e.g., dextrose); dispersants (e.g., Tween 20); and/or one or more components suitable as an alkalizing agent, buffering agent, emulsifier, or sequestering agent), and/or binding agent (e.g., sodium citrate).

The compositions of the invention are preferably suitable for preparation as pharmacological formulations. The compositions may be mixed with pharmaceutically-acceptable excipients which are compatible with the peptides and/or other active agents and are pharmaceutically acceptable. Suitable excipients may include water, saline, dextrose, glycerol, ethanol, and combinations thereof. The composition may further contain auxiliary substances such as wetting or emulsifying agents or pH buffering agents to enhance the administration and/or resulting treatment.

According to preferred embodiments, the therapeutic composition further comprises: (a) taste flavorants and/or flavorings such as mint, chocolate, citrus, etc. (e.g., for oral administration compositions) and/or (b) smell flavorants and/or flavorings such as citrus, floral etc. (e.g., for nasal administration compositions).

The compositions of this invention may be combined or mixed with various solutions and other compounds as is known in the art. For example, it may be administered in unit doses in water, saline or buffered vehicles.

Alternatively, such compositions may also be readily applied as a “spray”, which solidifies into a film or coating. Such sprays may be prepared from microspheres or particles of a wide array of sizes, including for example, from 0.1 μm to 3 μm, from 10 μm to 30 μm, and from 30 μm to 100 μm. For example, as described in U.S. Pat. No. 10,085,938 to Kovarik et al, hereby incorporated by reference in its entirety.

Another aspect of the invention relates to nasal spray formulations comprising a therapeutic composition according to the invention. That is, a possible prophylactic treatment which can be sprayed daily (or more often) to destroy any viruses within the nasal passages before they migrate to the throat and then the lungs, in addition to after the symptoms occur.

Preferably, the formulation and/or liquid is configured to be sprayed as an aerosol.

Preferably, the nasal spray formulations include the sprayable gel-type skin/mucosa-adhesive preparations described in WO2007123193 to Taizou; US20130316009 to Popov; AU2007241815 and U.S. Pat. No. 8,771,711 to Kamishita; and/or U.S. Pat. No. 9,993,425 to Costantino, wherein the therapeutic compositions described herein are used as the active agent or ingredient, each hereby incorporated by reference in its entirety.

Further embodiments of the invention relate to devices for administration of the therapeutic compositions described herein and comprising the compositions for administration. Devices for delivering nasal sprays are known. These are typically hand-held containers designed to hold fluids. The preferred container comprises two ends with one end being a flexible base and the second end being a tip having a blunt taper ending in an opening in communication with the fluid in the base. The blunt taper allows for partial insertion of the tip and opening into a nostril. When the flexible base is squeezed, a metered volume of aerosol fluid is delivered through an opening in the tip into the nose for inhalation and delivery to the nasal mucosal membranes.

Another aspect of the invention is to provide the compositions of the invention used to treat or prevent pathogen infectious diseases, particularly viral infectious diseases, or other illnesses or diseases or symptoms thereof, wherein the composition comprises binding molecule-surface active agent complexes suitable for the treatment and/or prevention of the infectious diseases wherein the binding molecules have effector molecules that are antibodies specific for one or more antigen on a pathogen and one or more surface active agents capable of catalyzing a reaction that destroys the infectivity of the pathogen of interest, e.g., hydrolysis of viral coat proteins or viral envelope lipids.

According one aspect of the invention, the therapeutic composition is applied as a prophylactic treatment which can be taken or administered daily (or more often) to destroy any pathogens (e.g., viruses) within the throat before they migrate to lungs, both before and after exposure to the pathogen, in addition to use as a treatment for the illness caused by the pathogen.

According to another embodiment, the therapeutic composition is incorporated into a cough drop or lozenge or chewing gum or lollipop or other edible product to slowly release the composition to coat the throat of the user. Preferably, the resulting liquid coats the throat with a film with the therapeutic components either within film matrix, for example, the particles and/or active agents (antibody and/or surfactant) dispersed within the film and/or on the film's surface, applied at least temporarily to the throat to destroy the pathogen (e.g., virus) while in the throat.

Preferably, the lozenge is a sequential release lozenge or gum used to slowly release the therapeutic components to act on the pathogens or foreign substance within the mouth and throat.

According to another embodiment, the therapeutic composition is incorporated into a film with active agents attached and the film is contacted to or attached to the throat or otherwise applied to a treatment area (e.g., swallowed partially and withdrawn). For example, such as compositions and formations described in U.S. Pat. No. 10,085,938 to Kovarik et al, and combined or altered with the therapeutic compositions of the invention, herein incorporated by reference in its entirety.

Preferably, the compositions described herein include one or more mucosa-adhesives (e.g., Korean Patent publication KR100413877B1 and U.S. Pat. No. 6,319,513 to Dobrozsi, hereby incorporated by reference in its entirety) which are employed to slowly release the formulation to act on viruses within mouth and throat and/or nasal passages.

Another aspect of the invention relates to methods for administering or using one or more the therapeutic compositions or formulations or products to treat a human or animal.

According to one embodiment, the invention relates to a method of treating or reducing the risk of at least one disease, illness or symptom(s) in a human, the method comprising administering in said human the therapeutic compositions or formulations or products that include one or more components that specifically binds to the pathogen (virus) or analyte of interest (e.g., allergen or other foreign substance).

According to one embodiment, the invention relates a method of inhibiting, treating or reducing the risk of at least one disease, illness or symptom(s), the method comprising administering to a subject in need thereof a therapeutically effective amount of the therapeutic compositions or formulations or products, as described herein, administered topically, by injection, to the eye, orally, or by inhalation.

According to one preferred embodiment, the invention relates a method of inhibiting, treating or reducing the risk of at least one disease, illness or symptom(s), the method comprising administering to a subject in need thereof a therapeutically effective amount of the therapeutic compositions or formulations or products, as described herein, administered orally.

According to another preferred embodiment, the invention relates a method of inhibiting, treating or reducing the risk of at least one disease, illness or symptom(s), the method comprising administering to a subject in need thereof a therapeutically effective amount of the therapeutic compositions or formulations or products, as described herein, administered by inhalation.

According to another preferred embodiment, the invention relates a method of inhibiting, treating or reducing the risk of at least one disease, illness or symptom(s), the method comprising administering to a subject in need thereof a therapeutically effective amount of the therapeutic compositions or formulations or products, as described herein, administered nasally (e.g., via nasal spray).

According to another preferred embodiment, the invention relates a method of inhibiting, treating or reducing the risk of at least one disease, illness or symptom(s), the method comprising administering to a subject in need thereof a therapeutically effective amount of the therapeutic compositions or formulations or products, as described herein, administered by application or administration of a film, paste or other composition to the upper respiratory system, wherein the film, paste or other composition comprises a therapeutic composition according to the invention. Preferably, the film, paste or other composition is applied or administered to the throat and/or to nasal passages. According to one preferred embodiment, the film, paste or other composition comprises a complex includes at least one active agent (e.g., surfactant) and at least one antibody or antibody fragment that specifically binds to the pathogen or foreign substances. Preferably, the at least one active agent (e.g., surfactant) is connected to the at least one antibody or antibody fragment, preferably directly connected or connected by a bifunctional linking reagent or linkage. According to an alternative preferred embodiment, the film, paste or other composition comprises at least one particle and at least one antibody structure comprising an antibody or antibody fragment that specifically binds to a foreign substance, wherein the at least one antibody structure is attached to, connected to, adsorbed onto or embedded in the at least one particle, wherein the particle has an aerodynamic diameter of greater than 10 μm and less than 100 mm.

Preferably, the method further comprises administering a flushing solution (e.g., saline) and/or a neutralizing composition/solution to the treatment area to remove the therapeutic composition and/or otherwise inactivate/neutralize the therapeutic composition after an effective amount of time for treatment.

Preferably, the method further comprises administering one or more other therapeutic compositions including anti-cough and/or anti-sneeze agents, as discussed herein.

This invention further provides for a pharmaceutical composition and/or therapeutic composition(s) comprising a therapeutically effective amount of the active agents, as described here. The pharmaceutical and/or therapeutic composition(s) can be formulated as a solution, a powder, a cream, a gel, an ointment, a douche, a suspension, a tablet, a pill, a capsule, a nasal spray, a nasal drop, a suppository and an aerosol. Alternatively, the pharmaceutical composition and/or therapeutic composition(s) can be formulated as a pessary, a tampon, a gel, a paste, a foam, and a spray.

Another embodiment relates to gels (including microgels), creams, pastes or other formulations comprising a therapeutic composition as described herein and, preferably, configured to be applied to a medical instrument or tool or component that will be inserted into an individual and/or otherwise contact an infected area requiring treatment (e.g., surgical instrument, catheter, or other medical device). For example, an endotracheal tube coated within a formulation comprising a therapeutic composition as described herein (e.g., as shown in FIG. 8).

In a specific embodiment, an effective amount of a therapeutic compositions (as described herein) or a prophylactic agent reduces one or more of the following infection steps: the docking of the virus particle to a cell, the introduction of viral genetic information into a cell, the expression of viral proteins, the production of new virus particles, the release of virus particles from a cell, and/or the docking of the released new virus particles to new cells, by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. In another specific embodiment, an effective amount of a therapeutic or a prophylactic agent reduces the replication, multiplication or spread of a virus by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

Preferably, the therapeutic composition is used to prevent or impede the onset, development, progression, and/or severity of an infection, illness or disease or a symptom thereof and/or reduce the likelihood of spreading the infection/disease to others. That is, for example, after exposure to infection, announcement of community spread, announcement of new infections or otherwise as preventive (e.g., seasonal). Alternatively, during allergy season, the therapeutic composition containing antibodies specific to allergens is administered. According to another embodiment, the therapeutic composition(s) is administered before anticipated exposure of the pathogen and/or foreign substance.

According to preferred embodiments, for treatment of the upper respiratory system (e.g., throat and/or nasal passages), the therapeutic composition is administered to a user in the evening before bedtime so the treatment can continue while the user is lying down horizontally after application to increase the likelihood the therapeutic composition remains in treatment area for a longer period of time.

According to preferred embodiments, the protocol for the treatment further comprises flushing the treatment area with saline solution(s) to remove the therapeutic composition after an effective time of treatment and/or with a neutralizing solution/composition as described above.

Yet another aspect of the invention relates to a method of using a therapeutic composition as described herein, the method comprising inserting an endotracheal tube (ET tube) through an individual's nose and/or mouth and into the individual's trachea, wherein the ET tube is coated with a gel or cream or other formulation including a therapeutic composition as described herein and/or the therapeutic composition is administered via the ET tube after the ET tube is inserted into the patient.

Another embodiment relates to a method comprising coating an ET tube with a gel or cream or other formulation including the therapeutic composition.

According an alternative embodiment, the ET tube has an outer surface and at least one antibody and/or at least one surface active agent (e.g., surfactant) is attached to, connected to, adsorbed onto, absorbed into and/or embedded in the outer surface of the ET tube. That is, the therapeutic composition is on the outer surface of the ET tube (e.g., the active agents immobilized on the outer surface, for example, using a film or gel).

Preferably, the at least one antibody and/or at least one surface active agent are configured to disrupt, destroy and/or otherwise inactive any viruses within the upper and/or lower respiratory system surfaces contacting the ET tube, as described above.

According to another embodiment, the method comprises inserting a laryngoscope blade through an individual's mouth and into the individual's trachea, wherein the laryngoscope blade has an outer surface and an interior channel for inserting an ET tube and the interior channel has an interior surface enclosing the interior channel, wherein the outer surface and/or interior channel and/or interior surface comprises the therapeutic composition (e.g., immobilized on surfaces) and/or is coated with a gel or cream or other formulation including the therapeutic composition and/or the therapeutic composition is introduced into the blade and/to ET tube during use or immediately prior to use. Preferably, the at least one antibody and/or at least one surface active agent are configured to disrupt, destroy and/or otherwise inactive any viruses within the internal areas of the body contacting the laryngoscope blade while being inserted and while inserted into the individual.

Preferably, a gel or a cream or other formulation coats at least the outer surface of the view tube or blade of laryngoscope and/or preferably also the interior of view tube (i.e., channel) so the inner ET tube that will be inserted through the channel and that will remain in trachea during the ventilation will be coated with the gel or cream or other formulation comprising the therapeutic composition.

According to another preferred embodiment, the inner ET tube that will remain in trachea during ventilation will be coated, with or without also coating the outer and insert of tube of laryngoscope.

According to additional embodiments, the method further comprises injecting or administering additional therapeutic composition to the ET tube while inserted in the throat. According to one preferred embodiment, the ET tube further comprises an outer tube surrounding the central ET tube providing an annular channel around the ET tube to allow the additional therapeutic composition to be injected and/or otherwise administered via the annular channel of the ET tube and the outer tube comprising pores or other openings to release the additional therapeutic composition within the treatment area without removing the ET tube from the individual.

Another embodiment of the invention relates to an ET tube having an outer annular channel for administering the therapeutic compositions as described herein and/or other medicines via pores or holes or other openings in the outer surface.

FIG. 8 is a graphical representation of an ET tube 800 according to one embodiment of the invention including an inner tube 801 having an inner channel 802 and an outer tube 803 providing an annular channel 804 between inner tube 801 and outer tube 803 configured for introducing, inserting and/or the administration of additional therapeutic composition and/or other medication and/or formulation. Outer tube 803 comprises a plurality of openings 806 on the outer surface 805 for administration of the additional therapeutic composition and/or other medication and/or formulation while ET tube 800 is within the tracheal.

Another aspect of the invention relates to a paste or gel or other composition comprising one or more of the therapeutic compositions described herein and configured for application to a wound, surgery location, or other surfaces (e.g., surface of implants) to inactivate, reduce and/or eliminate bacterial and/or viral infections or otherwise provide effective treatment for a disease, illness and/or symptom thereof.

According to one embodiment, the paste or gel or other composition is applied to a band aid, packings, pad, dressing, or bandage or gauze, or other material configured to be applied to a wound or post-surgery location or other location within body requiring treatment for a disease, illness and/or symptom thereof. Preferably, the paste or gel or other composition is applied to one surface of the dressing, for example, preferably the surface that contacts the wound or post-surgery area (see discussion of FIG. 9 below). According to an alternative embodiment, the paste or gel or other composition including the therapeutic composition is applied to the surface(s) of surgical implants before implantation to reduce the risk of infection.

According to preferred embodiments, the paste or gel or other composition comprises a plurality of particles, wherein the particles comprise antibodies and/or at least one surface active agent (e.g., surfactant) attached to, linked to, connected to, adsorbed onto, absorbed into and/or embedded thereto. Preferably, the particles are dissolvable after application to the wound or post-surgery location. Preferably, the particles are not bioactive when applied to wound or post-surgical area.

According to another embodiment, one or more therapeutic compositions are embedded in a band aid, packings, pad, dressing, or bandage or gauze or other material applied to wound or other location in the upper or lower respiratory system or elsewhere on the body. Preferably, the band aid, packings, pad, dressing, or bandage or gauze or other material comprises at least one antibody and/or at least one surface active agent (e.g., surfactant) attached to the material of the dressing or other item (or linked to, connected to, adsorbed onto, absorbed into and/or embedded thereto).

FIG. 9 shows wound dressing 900 including adhering side 902 comprising an adhesive region 901 (to facilitate adherence of dressing to the wound area) and centrally located therapeutic region 904. Therapeutic region 904 preferably comprises one or more gels or pastes or compositions comprising one or more of the therapeutic complexes described herein.

According to one embodiment the therapeutic region 904 comprises at least one antibody and/or at least one surface active agent (e.g., surfactant) attached to, linked to, connected to, adsorbed onto, absorbed into and/or embedded on the surface of therapeutic region 904.

According to another embodiment, the therapeutic region 904 comprises a plurality of the particles as described herein attached to, connected to, adsorbed onto, absorbed into and/or embedded thereto, wherein the particles comprises at least one antibody and/or at least one surface active agent (e.g., surfactant) as described herein.

One preferred embodiment relates to biodegradable materials (e.g., wound dressing or bandage material made of biodegradable materials and/or materials that dissolve in the body) comprising at least one antibody and/or at least one surface active agent (e.g., surfactant) attached to, connected to, adsorbed onto, absorbed into and/or embedded thereto.

Preferably, the therapeutic agent(s) are within a gel or film or solution or polymer or other composition applied to the material of the dressing, implant, etc.

According to another preferred embodiment, the materials (e.g., wound dressing or bandage material) are absorbent or include at least one absorbent layer to absorb the at least one antibody and/or at least one surface active agent or composition containing the same.

According to another preferred embodiment, the materials (e.g., wound dressing or bandage material) are adapted or configured for use as a nasal packing post-surgery (e.g., post endoscopic sinonasal surgery), more preferably the “material” is a gel or film or foam applied to the wound area to improve postoperative symptoms and mucosal healing after surgery as compared with conventional nasal packings.

The therapeutic compositions of the invention can be applied to or incorporated into any band aid, packings, pad, dressing, or bandage or gauze or other material configured to be applied to a wound or post-surgery location. See, for example, U.S. Pat. No. 5,584,827 to Korteweg et al.; U.S. Pat. No. 1,732,697 to Ryan; U.S. Pat. No. 2,179,964 to Stevens; U.S. Pat. No. 3,049,125 to Kriwkowitsch; U.S. Pat. No. 3,570,494 to Gottschalk; U.S. Pat. No. 4,030,504 to Doyle; U.S. Pat. No. 4,646,739 to Doyle; U.S. Pat. No. 4,568,326 to Rangaswamy; U.S. Pat. No. 4,950,280 to Brennan; U.S. Pat. No. 5,843,030 to Cercone; U.S. Pat. Pub. No. 20170172806 to Fung et al.; U.S. Pat. Pub. No. 20190350765 to Heagle et al.; U.S. Pat. Pub. No. 20190110932 to Mumby et al.; U.S. Pat. Pub. No. 20190314209 to Ha et al.; U.S. Pat. No. 6,685,682 to Heinecke et al; U.S. Pat. Pub. No. 20190008690 to Adle at al.; U.S. Pat. Pub. No. 20150313593 to Patenaude; U.S. Pat. Pub. No. 20180015197 to Cotton; U.S. Pat. No. 8,668,924 to McCarthy et al.; and U.S. Pat. No. 10,548,776 to Greener, each disclosing materials (e.g., absorbent materials, gels, foams, etc.) for use as packings or wound dressings and methods of using the same, each hereby incorporated by reference in its entirety.

According to one preferred embodiment, at least one antibody and/or at least one surface active agent is randomly attached to, connected to, adsorbed onto, absorbed into and/or embedded to the material of the dressing.

According to another preferred embodiment, at least one antibody and/or at least one surface active agent is independently or each individually attached to, connected to, adsorbed onto, absorbed into and/or embedded to the material (e.g., not collectively attached together to material). According to another preferred embodiment, at least one antibody is attached to, connected to, adsorbed onto, absorbed into and/or embedded to the material and at least one surface active agent is attached or linked to the at least one antibody, as shown in FIG. 3(b) (wherein particle 351 is replaced with material or surface of the bandage or other wound dressing). According to another preferred embodiment, at least one antibody is attached or linked to the at least one surface active agent forming a complex (as shown in FIG. 3(a)) and the complex is attached to, connected to, adsorbed onto, absorbed into and/or embedded to the material of the bandage or other wound dressing.

According to another preferred embodiment, at least one antibody and at least one surface active agent is attached to, connected to, adsorbed onto, absorbed into and/or embedded into at least one particle and the at least one particle is attached to, connected to, adsorbed onto, absorbed into and/or embedded into the material of the bandage or other wound dressing. Preferably, the at least one particle is concave (as shown in FIG. 4), an assemblage of fibers (as shown in FIG. 5) or the particle as described in FIG. 2 or otherwise described herein.

According to another embodiment, the active components (e.g., antibody and/or surface-active agent and/or particles) are on one side of the material of the bandage or other wound dressing. According to another preferred embodiment, the active components (e.g., antibody and/or surface-active agent and/or particles) are on both sides (e.g., front and back) of the material of the bandage or other wound dressing.

According to another embodiment, the active components (e.g., antibody and/or surface-active agent and/or particles) are dispersed throughout the material of the bandage or other wound dressing. According to another embodiment, the active components (e.g., antibody and/or surface-active agent and/or particles) are centrally located on the surface of the material of the bandage or other wound dressing and preferably surrounded at least partially be an adhesive surface to adhere the material to the wound area.

Another embodiment of the invention relates to therapeutic containers comprising one or more of the packings or dressings as described herein sealed within a container or package or bag or the like. Preferably, the container or package or bag is re-sealable. According to an alternative embodiment, the packing or dressing comprises a removable film or layer or other covering configured to protect the therapeutic composition before the packing or dressing is applied.

In the description above, for purposes of explanation only, specific nomenclature is set forth to provide a thorough understanding of the present disclosure. However, it will be apparent to one skilled in the art that these specific details are not required to practice the teachings of the present disclosure. Moreover, the various features of the representative examples and the dependent claims may be combined in ways that are not specifically and explicitly enumerated in order to provide additional useful embodiments of the present teachings. It is also expressly noted that all value ranges or indications of groups of entities disclose every possible intermediate value or intermediate entity for the purpose of original disclosure, as well as for the purpose of restricting the claimed subject matter. It is also expressly noted that the dimensions and the shapes of the components shown in the figures are designed to help to understand how the present teachings are practiced, but not intended to limit the dimensions and the shapes shown in the examples.

The scope of the present devices, systems and methods, etc., includes both means plus function and step plus function concepts. However, the claims are not to be interpreted as indicating a “means plus function” relationship unless the word “means” is specifically recited in a claim, and are to be interpreted as indicating a “means plus function” relationship where the word “means” is specifically recited in a claim. Similarly, the claims are not to be interpreted as indicating a “step plus function” relationship unless the word “step” is specifically recited in a claim, and are to be interpreted as indicating a “step plus function” relationship where the word “step” is specifically recited in a claim.

It is understood that the embodiments described herein are for the purpose of elucidation and should not be considered limiting the subject matter of the disclosure. Various modifications, uses, substitutions, combinations, improvements, methods of productions without departing from the scope or spirit of the present invention would be evident to a person skilled in the art.

1. A method of treating a subject to reduce allergy symptoms relating to at least one allergen, the method comprising nasally administering a therapeutically effective amount of the liquid formulation to the subject to reduce allergy symptoms relating to the at least one allergen, wherein the liquid formulation comprises a plurality of antibody structures dispersed in a liquid, wherein the plurality of antibody structures comprises an antibody or antibody fragment, wherein the plurality of antibody structures specifically bind to at least one allergen and wherein the liquid formulation is adapted for administering to the subject nasally to reduce at least one symptom caused by the least one allergen on the subject.

2. The method of claim 1, wherein the at least one allergen is selected from the group consisting of pollen, dust mites, dust mite parts, dust mite excretions, and pet dander.

3. The method of claim 1, wherein the liquid formation has a viscosity at 70° F. ranging from about 10 centipoise to about 100,000 centipoise.

4. The method of claim 1, wherein the liquid formation is thixotropic.

5. The method of claim 1, wherein the liquid formation is configured to be sprayed as an aerosol.

6. The method of claim 1, wherein the liquid formation comprises (i) water; (ii) at least one film forming agent; (iii) at least one emulsifier and/or thickening agent; and (iv) at least one antimicrobial agent.

7. A liquid formulation comprising a plurality of antibody structures dispersed in a liquid, wherein the plurality of antibody structures comprises an antibody or antibody fragment, wherein the plurality of antibody structures specifically bind to at least one allergen and wherein the liquid formulation is adapted for administering to a subject nasally to reduce at least one symptom caused by the least one allergen on the subject.

8. The liquid formulation of claim 7, wherein the at least one allergen is selected from the group consisting of pollen, dust mites, dust mite parts, dust mite excretions, and pet dander.

9. The liquid formulation of claim 7, wherein the liquid formation has a viscosity at 70° F. ranging from about 10 centipoise to about 100,000 centipoise.

10. The liquid formulation of claim 7, wherein the liquid formation has a viscosity at 70° F. ranging from about 500 centipoise to about 50,000 centipoise.

11. The liquid formulation of claim 7, wherein the liquid formation has a viscosity at 70° F. ranging from about 1000 centipoise to about 25,000 centipoise.

12. The liquid formulation of claim 7, wherein the liquid formation is thixotropic.

13. The liquid formulation of claim 7, wherein the liquid formation is configured to be sprayed as an aerosol.

14. The liquid formulation of claim 7, wherein the liquid formation comprises (i) water; (ii) at least one film forming agent; (iii) at least one emulsifier and/or thickening agent;
and (iv) at least one antimicrobial agent.

15. The liquid formulation of claim 7, wherein the liquid formation comprises water, saline, dextrose, glycerol, ethanol, or combinations thereof.

16. The liquid formulation of claim 7, wherein the liquid formation further comprises a dispersant.

17. The liquid formulation of claim 7, wherein the liquid formation further comprises anti-sneeze agents.

18. The liquid formulation of claim 7, wherein the plurality of antibody structures comprises first antibody structures specific to a first protein and second antibody structures specific to a second protein.

19. A liquid formulation comprising means for specifically binding at least one allergen, wherein the means for specifically binding is dispersed in a liquid, and wherein the liquid formulation is adapted for administering to a subject nasally to reduce at least one symptom caused by the least one allergen on the subject.

20. A nasal spray device comprising the liquid formulation of claim 7, wherein the nasal spray device comprises a base comprising a metered volume of the liquid formulation and a tip having an opening in communication with the liquid formulation in the base.
